SEQUENCE LISTING (64 sequences)

SEQ ID NO: 1 - DNA, 34 bp, Artificial Sequence
  Description of Artificial Sequence Synthetic primer
ggatccgaattcatggcgggacacctggcttcgg

SEQ ID NO: 2 - DNA, 60 bp, Artificial Sequence
  Description of Artificial Sequence Synthetic primer
aaaaaagtcgacgcggcgtctgcgtctgcggcgtctgcggtttgaatgcatgggagagcc

SEQ ID NO: 3 - DNA, 34 bp, Artificial Sequence
  Description of Artificial Sequence Synthetic primer
ggatccgaattcatgtacaacatgatggagacgg

SEQ ID NO: 4 - DNA, 59 bp, Artificial Sequence
  Description of Artificial Sequence Synthetic primer
aaaaaactcgaggcggcgtctgcgtctgcggcgtctgcgcatgtgcgacaggggcagtg

SEQ ID NO: 5 - DNA, 34 bp, Artificial Sequence
  Description of Artificial Sequence Synthetic primer
ggatccgaattcatggctgtcagcgacgcgctgc

SEQ ID NO: 6 - DNA, 63 bp, Artificial Sequence
  Description of Artificial Sequence Synthetic primer
aaaaaactcgaggcggcgtctgcgtctgcggcgtctgcgaaagtgcctcttcatgtgtaaggc

SEQ ID NO: 7 - DNA, 35 bp, Artificial Sequence
  Description of Artificial Sequence Synthetic primer
ggatccgaattcatgcccctcaacgttagcttcac

SEQ ID NO: 8 - DNA, 63 bp, Artificial Sequence
  Description of Artificial Sequence Synthetic primer
aaaaaactcgaggcggcgtctgcgtctgcggcgtctgcgcgcacaagagttccgtagctgttc

SEQ ID NO: 9 - DNA, 20 bp, Artificial Sequence
  Description of Artificial Sequence Synthetic primer
actctgaggaggaacaagaa

SEQ ID NO: 10 - DNA, 19 bp, Artificial Sequence
  Description of Artificial Sequence Synthetic primer
tggagacgtggcacctctt

SEQ ID NO: 11 - DNA, 20 bp, Artificial Sequence
  Description of Artificial Sequence Synthetic primer
tctcaaggcacacctgcgaa

SEQ ID NO: 12 - DNA, 20 bp, Artificial Sequence
  Description of Artificial Sequence Synthetic primer
tagtgcctggtcagttcatc

SEQ ID NO: 13 - DNA, 24 bp, Artificial Sequence
  Description of Artificial Sequence Synthetic primer
gacagggggaggggaggagctagg

SEQ ID NO: 14 - DNA, 26 bp, Artificial Sequence
  Description of Artificial Sequence Synthetic primer
cttccctccaaccagttgccccaaac

SEQ ID NO: 15 - DNA, 20 bp, Artificial Sequence
  Description of Artificial Sequence Synthetic primer
agctacagcatgatgcagga

SEQ ID NO: 16 - DNA, 20 bp, Artificial Sequence
  Description of Artificial Sequence Synthetic primer
ggtcatggagttgtactgca

SEQ ID NO: 17 - DNA, 20 bp, Artificial Sequence
  Description of Artificial Sequence Synthetic primer
tgaacctcagctacaaacag

SEQ ID NO: 18 - DNA, 20 bp, Artificial Sequence
  Description of Artificial Sequence Synthetic primer
tggtggtaggaagagtaaag

SEQ ID NO: 19 - DNA, 20 bp, Artificial Sequence
  Description of Artificial Sequence Synthetic primer
tgtgcaccaacatctacaag

SEQ ID NO: 20 - DNA, 20 bp, Artificial Sequence
  Description of Artificial Sequence Synthetic primer
gcgttcttggctttcaggat

SEQ ID NO: 21 - DNA, 20 bp, Artificial Sequence
  Description of Artificial Sequence Synthetic primer
tcgctgagctgaaacaaatg

SEQ ID NO: 22 - DNA, 20 bp, Artificial Sequence
  Description of Artificial Sequence Synthetic primer
cccttcttgaaggtttacac

SEQ ID NO: 23 - DNA, 20 bp, Artificial Sequence
  Description of Artificial Sequence Synthetic primer
aaatgtttgtgttgcggtca

SEQ ID NO: 24 - DNA, 20 bp, Artificial Sequence
  Description of Artificial Sequence Synthetic primer
tctggcacaggtgtcttcag

SEQ ID NO: 25 - DNA, 23 bp, Artificial Sequence
  Description of Artificial Sequence Synthetic primer
ctggcaccacaccttctacaatg

SEQ ID NO: 26 - DNA, 22 bp, Artificial Sequence
  Description of Artificial Sequence Synthetic primer
aatgtcacgcacgatttcccgc

SEQ ID NO: 27 - DNA, 20 bp, Artificial Sequence
  Description of Artificial Sequence Synthetic primer
caaagttgtcatggatgacc

SEQ ID NO: 28 - DNA, 18 bp, Artificial Sequence
  Description of Artificial Sequence Synthetic primer
ccatggagaaggctgggg

SEQ ID NO: 29 - DNA, 25 bp, Artificial Sequence
  Description of Artificial Sequence Synthetic primer
ttaggaaaatgggtagtagggattt

SEQ ID NO: 30 - DNA, 30 bp, Artificial Sequence
  Description of Artificial Sequence Synthetic primer
tacccaaaaaacaaataaattataaaacct

SEQ ID NO: 31 - DNA, 25 bp, Artificial Sequence
  Description of Artificial Sequence Synthetic primer
atttgttttttgggtagttaaaggt

SEQ ID NO: 32 - DNA, 27 bp, Artificial Sequence
  Description of Artificial Sequence Synthetic primer
ccaactatcttcatcttaataacatcc

SEQ ID NO: 33 - DNA, 27 bp, Artificial Sequence
  Description of Artificial Sequence Synthetic primer
ggatgttattaagatgaagatagttgg

SEQ ID NO: 34 - DNA, 25 bp, Artificial Sequence
  Description of Artificial Sequence Synthetic primer
cctaaactccccttcaaaatctatt

SEQ ID NO: 35 - DNA, 25 bp, Artificial Sequence
  Description of Artificial Sequence Synthetic primer
tagttgggatgtgtagagtttgaga

SEQ ID NO: 36 - DNA, 25 bp, Artificial Sequence
  Description of Artificial Sequence Synthetic primer
taaaccaaaacaatccttctactcc

SEQ ID NO: 37 - DNA, 25 bp, Artificial Sequence
  Description of Artificial Sequence Synthetic primer
agagataggagggtaagtttttttt

SEQ ID NO: 38 - DNA, 26 bp, Artificial Sequence
  Description of Artificial Sequence Synthetic primer
actcccacacaaactaacttttattc

SEQ ID NO: 39 - DNA, 30 bp, Artificial Sequence
  Description of Artificial Sequence Synthetic primer
gagttaaagagttttgtttttaaaaattat

SEQ ID NO: 40 - DNA, 30 bp, Artificial Sequence
  Description of Artificial Sequence Synthetic primer
tcccaaatctaataatttatcatatctttc

SEQ ID NO: 41 - DNA, 25 bp, Artificial Sequence
  Description of Artificial Sequence Synthetic primer
ttaatttattgggattataggggtg

SEQ ID NO: 42 - DNA, 24 bp, Artificial Sequence
  Description of Artificial Sequence Synthetic primer
aacaacaaaacctaaaaacaaacc

SEQ ID NO: 43 - DNA, 20 bp, Artificial Sequence
  Description of Artificial Sequence Synthetic primer
ttttggtattggccatccta

SEQ ID NO: 44 - DNA, 21 bp, Artificial Sequence
  Description of Artificial Sequence Synthetic primer
gaaaggaacagagagacaggg

SEQ ID NO: 45 - DNA, 20 bp, Artificial Sequence
  Description of Artificial Sequence Synthetic primer
attgccccaaaacttttttg

SEQ ID NO: 46 - DNA, 20 bp, Artificial Sequence
  Description of Artificial Sequence Synthetic primer
ttgaagaccagtctgggaag

SEQ ID NO: 47 - DNA, 24 bp, Artificial Sequence
  Description of Artificial Sequence Synthetic primer
aacagaaccaataggctatctatc

SEQ ID NO: 48 - DNA, 24 bp, Artificial Sequence
  Description of Artificial Sequence Synthetic primer
tacagtaaatcacttggtaggaga

SEQ ID NO: 49 - protein, 9 aa, Artificial Sequence
  Description of Artificial Sequence Synthetic peptide
RRRRRRRRR

SEQ ID NO: 50 - protein, 13 aa, Human immunodeficiency virus
GRKKRRQRRRPPQ

SEQ ID NO: 51 - protein, 16 aa, Drosophila melanogaster
RQIKIWFQNRRMKWKK

SEQ ID NO: 52 - protein, 8 aa, Artificial Sequence
  Description of Artificial Sequence Synthetic peptide
RRRRRRRR

SEQ ID NO: 53 - protein, 9 aa, Artificial Sequence
  Description of Artificial Sequence Synthetic peptide
KGKKKGKKK

SEQ ID NO: 54 - protein, 5 aa, Artificial Sequence
  Description of Artificial Sequence Synthetic peptide
ARKKK

SEQ ID NO: 55 - protein, 9 aa, Artificial Sequence
  Description of Artificial Sequence Synthetic peptide
GGKKKKKKK

SEQ ID NO: 56 - protein, 5 aa, Artificial Sequence
  Description of Artificial Sequence Synthetic peptide
RKKKK

SEQ ID NO: 57 - protein, 6 aa, Artificial Sequence
  Description of Artificial Sequence Synthetic peptide
RRKKKK

SEQ ID NO: 58 - protein, 7 aa, Artificial Sequence
  Description of Artificial Sequence Synthetic peptide
AGKKKKR

SEQ ID NO: 59 - protein, 5 aa, Artificial Sequence
  Description of Artificial Sequence Synthetic peptide
GRRRR

SEQ ID NO: 60 - protein, 8 aa, Artificial Sequence
  Description of Artificial Sequence Synthetic peptide
GRRRGKKK

SEQ ID NO: 61 - protein, 9 aa, Artificial Sequence
  Description of Artificial Sequence Synthetic peptide
KKKKKKKKK

SEQ ID NO: 62 - protein, 9 aa, Artificial Sequence
  Description of Artificial Sequence Synthetic peptide
RRRHKKKRK

SEQ ID NO: 63 - protein, 10 aa, Artificial Sequence
  Description of Artificial Sequence Synthetic peptide
RRRHHKKKRK

SEQ ID NO: 64 - protein, 9 aa, Artificial Sequence
  Description of Artificial Sequence Synthetic peptide
RRRRRKKKH

(19) United States
(12) Patent Application Publication
(10) Pub. No.: US 2012/0128655 A1
(43) Pub. Date: May 24, 2012

(54) INDUCED PLURIPOTENT STEM CELLS

(75) Inventors: Dohoon Kim, Belmont, MA (US); Chun-Hyung Kim, Lexington, MA (US); Kwang-Soo Kim, Lexington, MA (US)

(73) Assignee: The McLean Hospital Corporation

(21) Appl. No.: 13/260,897

(22) PCT Filed: Apr. 1, 2010
(86) PCT No.: PCT/US10/29704

§ 371 Date: Nov. 14, 2011

Related U.S. Application Data
(60) Provisional application No. 61/166,635, filed on Apr. 3, 2009.

(51) Int. Cl. C12N 5/071; A61K 8/98

(52) U.S. Cl. 424/115; 435/377

(57) ABSTRACT

The present invention concerns the delivery of certain reprogramming factor proteins into cells, such as differenti-atedsomatic cells, in order to induce the epi-genetic reprogramming of the cell so it becomes a pluripotent stem cell. The reprogramming factor protein(s) may be Sox2, Klf4, Oct3/4, c-Myc, Lin28, Nanog, or any protein with reprogramming (-enhancing) activity. These proteins may be linked recombinantly or chemically to a cell penetrating peptide that helps facilitate the introduction of these proteins into the target cell and may be preferably expressed in mammalian cells to maintain them in active forms. Accordingly, the present method of inducing pluripotent stem cell (iPS) formation avoids the use of viral or DNA-based expression vectors or the expression of reprogramming factor genes within target cells, which are known to be harmful to the host target cell and cause cancer.

### CROSS-REFERENCE TO RELATED APPLICATIONS

This application claims benefit of U.S. Provisional Application No. 61/166,635, filed Apr. 3, 2009, which is incorporated by reference in its entirety.

### STATEMENT OF GOVERNMENT-SPONSORED RESEARCH

This invention was made with government support under Grant Nos. MH48866 and DC006501 awarded by the National Institutes of Health. The government has certain rights in the invention.

### FIELD OF THE INVENTION

The present invention relates to the production and use of pluripotent cells.

### BACKGROUND OF THE INVENTION

A pluripotent stem cell has the potential to differentiate into any one of the three different types of germ layers: (1) endoderm cells which constitute the interior stomach lining, gastrointestinal tract, and the lungs; (2) mesoderm cells, which constitute muscle, bone, blood, urogenital cells; and (3) ectoderm cells, which constitute epidermal tissues and nervous system cells. Because of these properties, pluripotent stem cells offer a powerful way to remake or replenish cells in various cell replacement therapies. Pluripotent stem cells thus provide a unique way to obtain a renewable source of healthy cells and tissues, which can be useful for treating any of a number of diseases.

Pluripotent stem cells are typically isolated from human embryos that are a few days old. Cells from these embryos can be used to create pluripotent stem cell lines that can be grown indefinitely in the laboratory. Multipotent stem cell lines have also been developed from fetal tissue obtained from fetal tissue (older than 8 weeks of development). However, even though pluripotent stem cells can give rise to any fetal or adult cell types, they cannot by themselves divide and develop into a fetal or adult animal because pluripotent stem cells lack the potential to contribute to extraembryonic tissue, such as the placenta.

Nevertheless, under the right circumstances, a pluripotent stem cell that is isolated from an embryo can produce almost all of the cells in the body. Yet after this embryonic development stage is over, the stem cells no longer have this unlimited potential to develop into all cell types. Their pluripotency is thus lost and they can only become certain types of cells, which are terminally-differentiated cells. These terminally-differentiated cells, however, can be “reprogrammed” so that they revert back to a undifferentiated pluripotent state. This method of reprogramming produces “induced pluripotent stem cells” (iPS).

Induced pluripotent stem cells therefore are a type of pluripotent stem cell that is artificially derived from a differentiated cell, such as an adult somatic cell, by forcing the expression of certain genes in that differentiated cell, which effectively resets the genotype of the cell to that of a pluripotent state. Accordingly, iPS cells are believed to have many features in common with natural pluripotent stem cells, such as embryonic stem cells, with regard to the expression of certain stem cell genes and proteins, chromatin methylation patterns, doubling time, embryoid body formation, teratoma formation, viable chimera formation, and potency and differentiability.

iPS cells are typically derived by transfection of certain stem cell-associated genes (reprogramming factors; “RPFs”) into non-pluripotent cells, such as adult fibroblasts. Transfection is typically achieved through viral vectors, such as retroviruses. Transfected genes include the master transcriptional regulators Oct-3/4 (Pouf51) and Sox2, which are described herein as nuclear reprogramming factors. After about a month, small numbers of transfected cells begin to become morphologically and biochemically similar to pluripotent stem cells, and are typically isolated through morphological selection, doubling time, or through a reporter gene and antibiotic selection.

Four pluripotency nuclear reprogramming factors may be used to create iPS cells: Oct-3/4, SOX2, c-Myc, and Klf4. A preferred marker that is used to detect iPS is “Nanog” a gene that is important in embryonic stem cells, and a major determinant of cellular pluripotency. One problem is that these reprogramming factors, are oncogenic and/or tumor-related. Furthermore, the viral transfection systems used to insert the genes at random locations in the host's genome can also lead to undesirable oncogenic and tumorogenic growths because the integration of expression cassettes abnormally disrupts the host cell's genome.

Thus, the current methods for reprogramming differentiated cells require the use of viral vectors to express gene sequences that encode the transcription factors important for reprogramming. This is undesirable and problematic because viral vectors are known to integrate into host cell genome, which is harmful to the cell and the organism. Accordingly, the present invention provides a different, non-dangerous, and non-oncogenic method of producing iPS cells without use of ant DNA or viral vectors.

### SUMMARY OF THE INVENTION

One aspect of the present invention is a method for producing a pluripotent stem cell, comprising contacting a differentiated cell (e.g., a somatic cell) with at least one reprogramming factor protein wherein the reprogramming factor protein(s) causes the differentiated cell to dedifferentiate.

In one embodiment, the reprogramming factor is selected from the group consisting of Oct4, Sox2, c-Myc, Klf4, Nanog, and Lin28.

In one embodiment, the method further comprises contacting the differentiated cells, which have been exposed to a reprogramming factor, with any protein that has reprogramming activity or reprogramming-enhancing activity. In one embodiment, the method further comprises contacting the differentiated cells with at least one of an inhibitor of p53, p16(Ink4a), and p19(Arf), ERas, ECAT15-2, Tcl1, and beta-catenin, ECAT1, Esg1, Dnmt3L, ECAT8, Gdf3, Sox15, ECAT15-1, Fthl17, Sal14, Rex1, UTF1, Stella, Stat3, and Grb2.

In another embodiment, the reprogramming factor is linked to a cell penetrating peptide. In one embodiment, the reprogramming factor is chemically conjugated to the cell penetrating peptide or is recombinantly linked to the cell penetrating peptide as a fusion protein.

In one embodiment, the cell penetrating peptide is a peptide that comprises (a) at least nine contiguous lysine amino acid resides, (b) at least nine contiguous arginine amino acid residues; (c) at least nine residues of a mixture of lysine and arginine amino acids, or (d) the HIV-TAT protein or a fragment thereof.

In another embodiments the differentiated cell is contacted with the at least one reprogramming factor either in vitro or in vivo.

In one embodiment, the method comprises contacting said differentiated cell with an Oct4 reprogramming factor protein, wherein the Oct4 reprogramming factor protein comprises at least nine contiguous lysine resides or at least nine contiguous arginine residues, and wherein the presence of the Oct4 reprogramming factor protein in the differentiated cell causes the differentiated cell to become a pluripotent stem cell (i.e, and induced pluripotent cell; “iPS”).

In one embodiment, the method comprises contacting said differentiated cell with a Sox2 reprogramming factor protein, wherein the Sox2 reprogramming factor protein comprises at least nine contiguous lysine resides or at least nine contiguous arginine residues, and wherein the presence of the Sox2 reprogramming factor protein in the differentiated cell causes the differentiated cell to become a pluripotent stem cell (i.e, and induced pluripotent cell; “iPS”).

In one embodiment, the method comprises contacting said differentiated cell with a c-Myc reprogramming factor protein, wherein the c-Myc protein comprises at least nine contiguous lysine resides or at least nine contiguous arginine residues, and wherein the presence of the c-Myc reprogramming factor protein in the differentiated cell causes the differentiated cell to become a pluripotent stem cell (i.e, and induced pluripotent cell; “iPS”).

In one embodiment, the method comprises contacting said differentiated cell with a Klf4 reprogramming factor protein, wherein the Klf4 reprogramming factor protein comprises at least nine contiguous lysine resides or at least nine contiguous arginine residues, and wherein the presence of the Klf4 reprogramming factor protein in the differentiated cell causes the differentiated cell to become a pluripotent stem cell (i.e, and induced pluripotent cell; “iPS”).

In another embodiment, the method comprises contacting said differentiated cell with an Oct4 conjugate, a Sox2 conjugate, a c-Myc conjugate, and a Klf4 conjugate, wherein each of said conjugates comprises a cell penetrating peptide, wherein said conjugates cause said cell to become a pluripotent stem cell (i.e, and induced pluripotent cell; “iPS”).

In one embodiment, the cell penetrating peptide comprises a plurality of substituents selected from amines, guanidines, amidines, N-containing heterocycles, or combinations thereof.

In another embodiment, the cell penetrating peptide comprises a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof.

In one embodiment, the cell penetrating peptide is an oligomer selected from the group consisting of oligopeptide, oligoamide, cationically functionalized oligoether, cationically functionalized oligosaccharide, oligoamine, oligoethyleneimine, and combinations thereof. In another embodiment, the oligomer is an oligopeptide.

In one embodiment, substantially all of the amino acid residues of the oligopeptide are capable of forming positive charges. In another embodiment, the oligopeptide comprises 5 to 15 amino acids. In another embodiment, the oligopeptide comprises 5 to 10 amino acids. In a further embodiment, the oligopeptide comprises at least nine contiguous lysine resides, at least nine contiguous arginine residues; or combinations thereof.

In another embodiment, the differentiated cells is contacted with the reprogramming factor conjugate protein either in vitro or in vivo.

Another aspect of the present invention is a reprogramming factor protein, comprising a cell penetrating peptide linked to a reprogramming factor selected from the group consisting of Oct4, Sox2, c-Myc, Lin28, Nanog, and Klf4. The present invention is not limited to only these particular reprogramming factors; other reprogramming factors may be used in accordance with the methods and techniques disclosed herein for producing iPS cells.

In one embodiment, the cationic conjugate comprises a plurality of substituents selected from amines, guanidines, amidines, N-containing heterocycles, or combinations thereof. In another embodiment, the cationic conjugate comprises a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof.

In one embodiment, the cationic conjugate is an oligomer selected from the group consisting of oligopeptide, oligoamide, cationically functionalized oligoether, cationically functionalized oligosaccharide, oligoamine, oligoethyleneimine, and combinations thereof.

In one embodiment, the oligomer is an oligopeptide. In one embodiment, substantially all of the amino acid residues of said oligopeptide are cationic. In another embodiment, the oligopeptide comprises 5 to 15 amino acids. In another embodiment, the oligopeptide comprises 5 to 10 amino acids. In one embodiment, the oligopeptide comprises at least nine contiguous lysine resides, at least nine contiguous arginine residues; or combinations thereof.

Another aspect of the present invention provides induced pluripotent stem cells produced from any of the methods disclosed herein.

Another aspect of the present invention provides differentiated cells produced from induced pluripotent stem cells made by any of the methods disclosed herein. In one embodiment, the differentiated cells are produced by (A) producing embryonic bodies from the induced pluripotent stem cells made using any of the methods described herein, and (2) incubating the embryonic bodies on ITSFn media, wherein the embryonic bodies differentiate into the cellular morphology of at least one germ layer selected from the group consisting of endoderm germ later cells, mesoderm germ layer cells, and ectoderm germ layer cells.

Another aspect of the present invention is a method for making differentiated cells, comprising (A) producing embryonic bodies from the induced pluripotent stem cells made using any of the methods described herein, and (2) incubating the embryonic bodies on ITSFn media, wherein the embryonic bodies differentiate into the cellular morphology of at least one germ layer selected from the group consisting of endoderm germ later cells, mesoderm germ layer cells, and ectoderm germ layer cells. In one embodiment, the differentiated cells are selected from the group consisting of neural cells (e.g., neurons, astrocytes, oligodendrocytes, or Schwann cells), epidermal cells, striated muscle cells, adipose cells, epithelial cells (e.g., respiratory epithelial cells or skin epithelial cells), fibroblasts (e.g., skin fibroblasts), and cornea-like epithelial cells.

Another aspect of the present invention is a method for treating an individual with a disease or injury, comprising (A) producing pluripotent stem cells by contacting differentiated cells from an individual with at least one reprogramming factor protein selected from the group consisting of Oct4, Sox2, c-Myc, and Klf4, wherein the presence of the reprogramming factor protein(s) in the individual's differentiated cells induces development of a pluripotent stem cell; (B) producing differentiated germ layer cells from the induced pluripotent stem cells; and (C) administering the differentiated germ layer cells to the individual, wherein the differentiated germ layer cells are disease-free and useful for treating the individual's disease or injury. In one embodiment, the disease or injury is a neurodegenerative diseases, leukemia, lymphoma, type 1 diabetes, a traumatic brain and/or spinal cord injury, degenerative spinal cord injuries and diseases, an injury to cardiac muscle, an injury to skeletal muscle, or an injury to skin. In another embodiment, the neurodegenerative disease is Parkinson's disease, Huntington's disease, or Alzheimer's disease; the degenerative spinal cord injury or disease is amyotrophic lateral sclerosis; the injury to the cardiac muscle is ischemia damage; and the injury to skin are lacerations, chemical burns, and thermal burns.

Another aspect of the present invention is a method for treating an individual with a disease or injury, comprising administering to said individual pluripotent stem cells made by any of the methods disclosed herein. In one embodiment, the individual has a neurodegenerative diseases, leukemia, lymphoma, type 1 diabetes, a traumatic brain and/or spinal cord injury, degenerative spinal cord injuries and diseases, an injury to cardiac muscle, an injury to skeletal muscle, or an injury to skin. In one embodiment, the neurodegenerative disease is Parkinson's disease, Huntington's disease, or Alzheimer's disease; the degenerative spinal cord injury or disease is amyotrophic lateral sclerosis; the injury to the cardiac muscle is ischemia damage; and the injury to skin are lacerations, chemical burns, and thermal burns.

An aspect of the present invention is a pharmaceutical composition, comprising pluripotent stem cells made by any of the methods disclosed herein.

A “pluripotent stem cell” is an undifferentiated cell that is capable of differentiating into various cell types of all three germ layers and is capable of in vitro self-replication, or self-renewal, for multiple generations and possibly an indefinite period of time, wherein the resultant daughter cells retain the undifferentiated characteristics of the parent cell. Pluripotent cells include embryonic stem cells but, other examples of pluripotent cells include induced pluripotent cells (see, for example, Takahashi et al., Cell, 126: 663-676, 2006; Cell, 131: 861-872, 2007; and Nakagawa et al., Nat. Biotechnol. 26: 101-106, 2008), pluripotent cells derived by nuclear transfer.

### BRIEF DESCRIPTION OF THE DRAWINGS

FIG. 1: Cellular uptake of reprogramming proteins fused with 9 repeat arginine as a cell-penetrating peptide: (a) Schematic representation of the mammalian expression vector of reprogramming factors. Each of KLF4, c-MYC, OCT4, and SOX2 cDNA was connected with the 9 repeat arginine as a cell-penetrating peptide and myc tagging peptide at C-terminus; (b) Human newborn fibroblast (HNF) cells were incubated with cell extracts from HEK 293 cells expressing each reprogramming protein at 37° C. for 8 hours and were subjected to immunocytochemistry using myc antibodies. Nuclei were counterstained with DAPI.

FIG. 2: Stable expression of reprogramming factors in HEK293 cells. HEK293 cells were transfected with pCMV hKLF4-9xArg-myc, pCMV hOCT4-9xArg-myc, pCMV hSOX2-9xArg-myc, and pCMV hc-MYC-9xArg-myc vectors, respectively and were grown in the presence of G-418 to select the stably transformed cells. 50 μg of cell lystaes protein were subjected to SDS-PAGE followed by western blotting using an anti-myc antibodies.

FIG. 3: Fluorescence microscopy analysis of COS-7 and HNF cells treated with dsRED and dsRED-9R proteins. a, COS-7 cells and b, HNF cells were treated with the extract of HEK 293 cells expressing dsRED (upper panel) and dsRED fused with 9 repeat arginine (dsRED-9R; lower panel) for 8 hours and were shown in 594 nm fluorescence.

FIG. 4: Various protocols for induction of p-iPS cells from human newborn fibroblasts.

FIG. 5: Generation and characterization of rv-hiPS01 cell line derived from human newborn fibroblasts (HNFs) using retroviral factors. a, Immunofluorescence staining of rv-hiPS01 clone shows expression of human ES makers including TRA-1-60, Oct-4, and SSEA-4. b, Embryoid body (EB)-mediated differentiation of rv-hiPS01 cells. EBs are made by suspension culture of rv-iPS cells at day 8. Phase contrast images show all three germ cells differentiated from EBs at day 24 including neural cells (ectodermal), endothelial-like cells (mesodermal), and endoderm-like cells (endoderm).

FIG. 6: Induction and characterization of p-hiPS (01 and 02) cell lines derived from human newborn fibroblasts (HNFs) using a novel protocol for p-iPS cell generation. a, The protocol shows a repeated process for generation of p-iPS cells from HNFs (first image of top panel). b, Protein treated-HNFs were changed in their morphology from 3 cycle repeats (second image of top panel) and repeated processes induce more colony formations after 6 cycles (third image of top panel). These colonies are clearly stained with alkaline phosphatase (AP) (fourth image of top panel). AP-positive colonies form two independent human ES cell lines with similar ES-like morphology on the mouse embryonic fibroblast (MEF). AP staining of formed iPS cell-like colonies (fourth image of top panel), the early morphology of p-iPS colonies derived from AP-stained iPS cell-like colonies at high magnification (fifth image of top panel), typical morphology of established p-iPS cell line at passage number 10 (second image of top panel). Immunofluorescence staining of p-hiPS01 clone (second panel) and p-hiPS02 clone (bottom panel) show expression of human ES makers including AP staining, SSEA-3, SSEA-4, Oct-4, Nanog, and TRA-1-60. Nuclei were stained with 4,6-Diamidino-2-phenylindole (DAPI) (blue at second and third row panel). c, Efficiency of iPS-like colony formation.

FIG. 7: Gene expression and epigenetic status in protein-induced human iPS cells. a, Quantitative RT-PCR was performed to asses the expression of c-MYC, GDF3, KLF4, NANOG, OCT4, REX1, SOX2, and hTERT, in protein-induced human iPS, H9, and HNF cells. Fold change represents the relative gene expression in HNF cells normalized to β-actin expression. This experiment has been repeated twice in triplicate using independently prepared cDNAs. b, Bisulfite sequencing analysis of the promoter region of NANOG and OCT4 in protein-induced iPS, H9, and HNF cells. Open and closed circles indicate unmethylated and methylated, respectively. Numbers in the top show CpG location relative to the transcription start site. Percentages of CpG methylation (% Me) were shown in the right of each panel.

FIG. 8: Expression of hES marker genes in HNF, H9, protein-induced iPS hiPS01 and p-hiPS02), and retrovirus-induced iPS (rv-hiPS01) cells. RT-PCR was performed using different primers to discriminate the relative expression of total, endogenous, and retrovirally expressed genes. β-Actin is used for an amplification control.

FIG. 9: DNA fingerprinting analysis. Genomic DNAs were prepared from protein-induced iPS, retrovirus-induced iPS, H9, and HNF cells. Genomic PCR was performed using the primer sets containing a highly variable of tandem repeats (Table 3). Amplified PCR products were subjected to 7% acrylamide gel electrophoresis.

FIG. 10: Embryoid body (EB)-mediated differentiation of p-hiPS (01 and 02) cells in vitro and in vivo differentiation. a, EBs are made by suspension culture of two p-iPS cell lines at day 8 (Left at top row panels). Phase contrast images (Top row panels) and immunostaining images (second and third row panels) show all three germ cells differentiated from EBs at day 24 including neural cells (ectodermal), muscle (mesodermal) endothelial-like cells (mesodermal), and endoderm-like cells (endoderm). b, Hematoxylin and eosin staining of teratoma derived from p-hiPS cells (01 and 02). The tumors are well-developed from a single injection site after cells were transplanted under the kidney capsules of SCID mice. The resulting teratomas demonstrate the following features representing ectoderm, mesoderm and endoderm differentiation (fourth row panel (p-hiPS01) and bottom row panel (p-hiPS02). Ectoderm: pigmented retinal cells and neural tissue; mesoderm: muscle and adipocyte, endoderm: respiratory epithelium and gut-like epithelium.

FIG. 11: Scheme 1 illustrates exemplary reprogramming factor protein linked with cationic conjugate at C or N terminus of the protein. In the exemplary conjugated proteins, the polypeptides comprise alpha amino acids, e.g. glycine, alanine, lysine, arginine or histidine, or beta amino acids, e.g. beta-alanine.

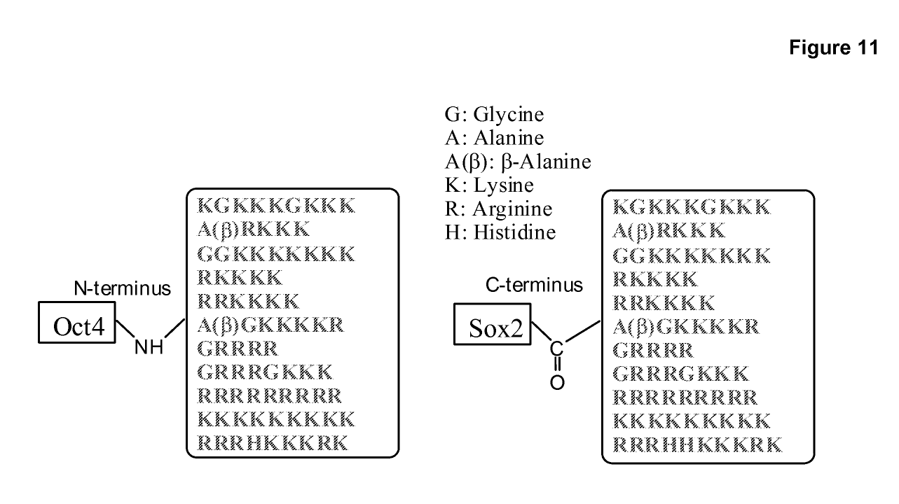

FIG. 12: Scheme 2: illustrates exemplary reprogramming factor protein linked with cationic conjugate via the amino acid side chain of the protein with or without linkers. The exemplary conjugated reprogramming factor proteins coupled with a cationic conjugate (a polypeptide or a polyamine) from its amino acid side chain directly or indirectly via a disulfide or urea linkage.

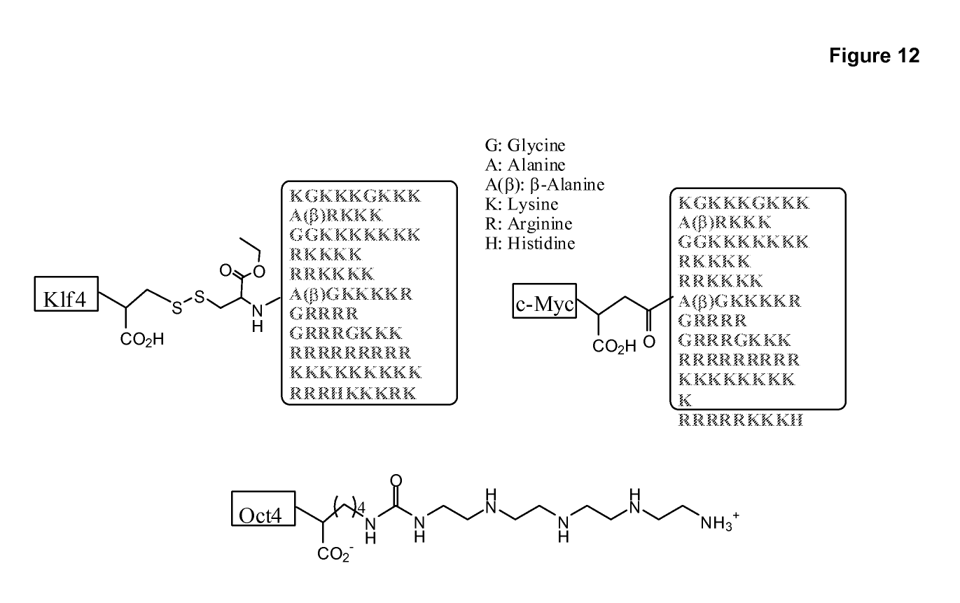

### DETAILED DESCRIPTION

The present invention provides compositions of pluripotent stem cells derived from somatic cells, including biopsy fibroblasts, and methods for producing the same. The pluripotent stem cells are provided as relatively substantially homogeneous populations having defined characteristics. A substantially homogenous cell population is a population or sample of cells which contain a majority (i.e., >50%) of cells having the desired phenotype (i.e., trait(s) of interest). In preferred embodiments, substantially homogenous populations contain at least 60%, at least 70%, at least 80%, at least 90%, or more of the cells having the desired phenotype.

The reprogrammed pluripotent cell of the present invention, referred to herein as an induced pluripotent stem cell (iPS), can then be cultured to develop and differentiate into a particularly desirable cell type under appropriate culture conditions. Methods for cell culturing, developing, and differentiating pluripotent stem cells may be carried out with reference to standard literature in the field. Suitable techniques are described by Wiles et al., Meth. Enzymol. 225:900, 1993; and in Embryonic Stem Cells (Turksen ed., Humana Press, 2002). Established protocols for generation, passaging and preservation of rodent and human pluripotent stem cells are described by, for example, Iannaccone et al. (Dev. Biol. 163:288, 1994); Matsui et al. (Cell 70:841, 1992), Thomson et al., Science 282:114, 1998; Shamblott et al., Proc. Natl. Acad. Sci. USA 95:13726, 1998; Reubinoff et al., Nat. Biotech. 18:399, 2000; U.S. Pat. Nos. 5,843,780 and 6,090,622; and PCT Publication Nos. WO 99/27076 and WO 00/27995. One other form of culturing step entails the use of a feeder cell layer such as a fibroblast feeder cell layer to culture the iPS cells.

The present invention also provides a method for preparing clinically feasible cell sources for treatment of human diseases, such a neurodegenerative diseases, in an individual by administering to them a composition or formulation containing iPS cells generated by the methods of the present invention. Diseases and disorders amenable to treatment using the therapeutic compositions and cells of the invention include, for example, neurodegenerative diseases (e.g., Parkinson's disease, Huntington's disease, and Alzheimer's disease), leukemia, lymphoma, type 1 diabetes, traumatic brain and/or spinal cord injury, degenerative spinal cord injuries and diseases (e.g., amyotrophic lateral sclerosis), injuries to the cardiac muscle (e.g., following ischemia damage such as a myocardial infarction, or traumatic injury), injuries to the skeletal muscle, and injuries to the skin (e.g., lacerations, chemical burns, and thermal burns).

In other embodiments, the cells contained in the therapeutic composition are encapsulated. See the subsection below on Genetic Diseases where this embodiment of the present invention is explained in more detail. The therapeutic compositions may be administered to the patient by any appropriate, known route.

As used herein, “cells derived from an iPS cell” refers to cells that are either pluripotent or terminally differentiated as a result of the in vitro culturing or in vivo transplantation of iPS cells.

Isolation Of Differentiated Cells

The types of somatic and differentiated cells to be reprogrammed according to the present invention to become iPS cells are not particularly limited, and any kinds of somatic cells may be used. For example, matured somatic cells may be used, as well as somatic cells of an embryonic period, as well as fibroblasts, hepatocytes, and gastric mucous cells. Indeed, any cell types from (1) endoderm cells, such as cells of the interior stomach lining, gastrointestinal tract, and the lungs; (2) mesoderm cells of muscle, bone, blood, urogenital cells; and (3) ectoderm cells of epidermal tissues and nervous system cells, may be manipulated according to the present invention and exposed to the reprogramming proteins disclosed herein in order to produce iPS cells.

The differentiated cell may be any mammalian cell, for example a mouse, human, rat, bovine, ovine, horse, hamster, dog, guinea pig, or ape cell. For example, direct reprogramming of such somatic cells provides an opportunity to generate individual- or disease-specific pluripotent stem cells. Mouse iPS cells are virtually indistinguishable from ES cells in morphology, proliferation, gene expression, and teratoma formation. Furthermore, when transplanted into blastocysts, mouse iPS cells can give rise to adult chimeras, which are competent for germline transmission (Maherali et al., Cell Stem Cell 1:55-70, 2007; Okita et al., Nature 448:313-17, 2007; Wemig et al., Nature 448:318-324, 2007). Human iPS cells are also expandable and virtually indistinguishable from human embryonic stem (ES) cells in morphology and proliferation. Furthermore, these cells can differentiate into cell types of the three germ layers in vitro and in teratomas.

In a clinical setting, informed and express consent from a human individual from whom cells are to be obtained directly may be required, such as by needle aspiration withdrawal of tissue and cells, or from any biological sample, such as a blood sample, tissue sample, saliva sample, hair sample, or semen sample, or any bodily fluid sample. See Aalto-Setala, et al., PLoS Biology, vol. 7(2), pp.: 204-208 (February 2009), on issues concerning informed consent.

Alternatively, human somatic cells may be obtained from a biological depository such as the ATCC and cultured according to the accompanying conditions provided with the deposit.

Reprogramming Factors

The reprogramming factors that may be delivered directly to and into cells of the present invention include, but are not limited to families of proteins expressed from genes of the Oct4 family, Sox2 family, Klf4 family, and c-myc family. See Yamanaka, “Strategies and New Developments in the Generation of Patient-Specific Pluripotent Stem Cells,” Cell Stem Cell 1:39-49 (July 2007), which is incorporated herein by reference.

Briefly, Oct4 is a transcription factor belonging to the Oct family and is specifically expressed in EC cells, early embryos, and germ cells. The Oct transcription factors contain a POU domain, which is about 150 amino acid region, that binds to the octamer sequence ATTA/TGCAT. Oct4 plays an important role in promoting cellular differentiation, and particularly so in neural and cardiac cell differentiation in mice. For clinical purposes of iPS cells, it is desirable to use human Oct4 cDNA sequences as a fused form with CPP and/or other sequences such as histidine repeat for purification purposes.

Sox2 is a Sox (SRY-related HMG box) protein which contains a high mobility group (HMG) domain that binds to DNA by recognizing the binding motif A/TA/TCAAA/TG. Sox2 is expressed in the ICM, epiblast, and germ cells and is also expressed by the multipotential cells of the extraembryonic ectoderm. It is associated with uncommitted dividing stem and precursor cells of the central nervous system. For clinical purposes of iPS cells, it is desirable to use human Sox2 cDNA sequences as a fused form with CPP and/or other sequences such as histidine repeat for purification purposes.

Klf4 is a Krüppel-like factor, zinc-finger protein that is highly expressed in differentiated, postmitotic epithelial cells of the skin and gastrointestinal tract. Klf4 is also expressed in fibroblasts and in undifferentiated mouse ES cells. Kl14 functions as a tumor suppressor and Oncogene. For clinical purposes of iPS cells, it is desirable to use human Klf4 cDNA sequences as a fused form with CPP and/or other sequences such as histidine repeat for purification purposes.

c-Myc binds, via its N-terminus, to several proteins, particularly to histone complex components. The C-terminus of c-Myc contains a basic region/helix-loop-helix/leucine zipper domain which binds c-Myc to its partner protein Max. The c-Myc-Max dimers bind to DNA sequences with the motif CACA/GTG, which exists throughout the human genome. Binding of the c-Myc-Max dimer can therefore modify chromatin structure and regulate gene expression. A problem with c-Myc is that it is known to induce oncogenesis. For clinical purposes of iPS cells, it is desirable to use human c-Myc cDNA sequences as a fused form with CPP and/or other sequences such as histidine repeat for purification purposes.

Each of such nuclear reprogramming proteins may be used alone or in combination with other nuclear reprogramming proteins as disclosed herein. In addition, a reprogramming protein of the present invention may be used in conjunction with small molecules, compounds, or other agents in order to obtain iPS cells.

In addition to these reprogramming factor proteins, other proteins can be co-delivered to the somatic or differentiated cell to help promote or enhance the induced pluripotent stem cell genotype, such as but not limited to Lin28, Nanog, ERas, ECAT15-2, Tcl1, and .beta.-catenin. Nanog is particularly useful for promoting pluripotency. Similarly, the following proteins also can be delivered to the differentiated cell in conjunction with one or more of the reprogramming factor proteins (e.g., Oct4 protein, Sox2 protein, Klf4 protein, and c-myc protein): ECAT1, Esg1, Dnmt3L, ECAT8, Gdf3, Sox15, ECAT15-1, Fthl17, Sal14, Rex1, UTF1, Stella, Stat3, and Grb2. In particular, protein factors that are specifically or preferentially expressed in oocyte and/or ES cells could be useful reprogramming factors. These factors can be used with or without one or more of the reprogramming factor proteins (e.g., Oct4 protein, Sox2 protein, Klf4 protein, and c-myc protein): ECAT1, Esg1, Dnmt3L, ECAT8, Gdf3, Sox15, ECAT15-1, Fthl17, Sal14, Rex1, UTF1, Stella, Stat3, and Grb2.

Along these lines, another embodiment of the present invention therefore includes the use of any protein with reprogramming activity or reprogramming-enhancing activity to help enhance or promote creation of a pluripotent stem cell. For example, recent studies showed that dominant negative form of p53 or an inhibitor of p53 increases the reprogramming efficiency (Hong et al., 2009, Nature, 460, 1132; Utikal et al., 2009, Nature, 460, 1145).

Thus, one embodiment comprises exposing cells, such as differentiated cells, to a dominant negative form of p53 or to an inhibitor of p53. Another embodiment comprises co-administering one of the reprogramming factors disclosed herein with a dominant negative form of p53 or with an inhibitor of p53, such a short hairpin RNA molecule (Hong et al.). Primary cell populations with low endogenous levels of active Trp53 (i.e., p53), p16(Ink4a), and p19(Arf) may also be useful for producing iPS cells at high efficiency (Utikal et al.)

Hence, one embodiment of the present invention contemplates exposing cells to combinations of (1) at least one of Oct4, Sox2, c-Myc, Klf4, Nanog, and Lin28, and (2) a dominant negative form of p53 or an inhibitor of p53, or in conjunction with any protein reprogramming activity or reprogramming-enhancing activity, as described herein.

Production of Reprogramming Factors

(A) Isolation and Purification from Cell Extracts

Reprogramming factor proteins of the present invention may be extracted, isolated, and purified from tissue samples or cell culture samples. Klf4, for instance, is highly expressed in differentiated, postmitotic epithelial cells of the skin and gastrointestinal tract. Hence, it is reasonable to obtain skin and gastrointestinal tract tissue samples from which Klf4 could be extracted and purified. Similarly, the Sox2 is expressed in the ICM, epiblast, and germ cells and multipotential cells of the extraembryonic ectoderm. Hence, these tissue types provide a source of Sox2 proteins. Likewise, Oct4 proteins may be isolated from neural and cardiac cells. There are various standard techniques for isolating proteins from cells, tissues, and cell extracts.

(B) Recombinant Production

A reprogramming factor of the present invention may be produced recombinantly by expressing the gene sequence, or a functional part thereof, in a cell. An cell expression system may be used to express a nucleotide encoding a functional factor, such as a human cell expression system, chicken hamster ovary cell expression system (CHO), or in insect cells or bacterial cells, for example. It could be desirable to use a mammalian cell expression system to express a reprogramming factor or another enhancing/promoting protein, e.g., nanog, to ensure the resultant expressed protein is properly or authentically folded and formed so as to maximize the activity of the expressed protein.

It is possible to create an expression cassette in which a reprogramming factor nucleotide sequence is operably linked to regulatory elements such as a promoter and terminator, which then can be expressed in one of these cell systems to produce the reprogramming factor protein.

Thus, an expression cassette of the present invention may comprise a polynucleotide sequence encoding any one of an Oct4, Sox2, c-Myc, Nanog, Lin28, and Klf4, operably linked to a promoter and appropriate polyadenylation and/or termination signal sequences. In one embodiment, an expression cassette of the present invention comprises a polynucleotide encoding Oct4, or a functional part thereof, operably linked to suitable regulatory elements that facilitate expression of Oct4 in a cell. In one embodiment, an expression cassette of the present invention comprises a polynucleotide encoding Sox2, or a functional part thereof, operably linked to suitable regulatory elements that facilitate expression of Sox2 in a cell. In one embodiment, an expression cassette of the present invention comprises a polynucleotide encoding c-Myc, or a functional part thereof, operably linked to suitable regulatory elements that facilitate expression of c-Myc in a cell. In one embodiment, an expression cassette of the present invention comprises a polynucleotide encoding Nanog, or a functional part thereof, operably linked to suitable regulatory elements that facilitate expression of Nanog in a cell. In one embodiment, an expression cassette of the present invention comprises a polynucleotide encoding Klf4, or a functional part thereof, operably linked to suitable regulatory elements that facilitate expression of Klf3 in a cell. In another embodiment, an expression cassette of the present invention comprises a polynucleotide encoding Lin28, or a functional part thereof, operably linked to suitable regulatory elements that facilitate expression of Lin28 in a cell.

A nucleotide sequence that encodes a “functional part” of one of these reprogramming transcription factors is one that encodes that part of the protein sequence responsible for interacting with genomic DNA or with other transcription factors, such as those parts of the factor that recognize particular DNA binding domains or are responsible for homo- or hetero-dimerization.

Thus, another embodiment of the present invention entails making an expression cassette in which a polynucleotide sequence that encodes a functional part of any one of Oct4, Sox2, c-Myc, Nanog, and Kf4, that is operably linked to regulatory sequences, such as to a promoter and appropriate polyadenylation and/or termination signal sequences.

The present invention also provides the expression of more than one reprogramming factor using recombinant expression techniques. Thus, an expression vector may comprise two or more expression cassettes, each of which expresses a different reprogramming factor. Accordingly, one transformation event may express two or more different reprogramming transcription factors.

A recombinantly expressed factor of the present invention may then be purified from the extract of the cell expression system according to standard techniques.

Production of Reprogramming Factor Fusion Proteins and Conjugates

To help facilitate the delivery of a reprogramming factor protein into a cell and across the cell membrane, the reprogramming factor protein, whether it be purified from a tissue or cell extract, or made recombinantly, may be fused chemically or recombinantly, or otherwise associated with a cationic conjugate, such as a cationic lysine-rich or arginine-rich peptide, or with other cationic conjugates as described below. The cationic conjugate may linked to the protein directly or via an appropriate linkers under suitable conditions.

In one aspect, the present invention is the creation of a fusion protein, for instance by chemical conjugation or recombinantly, between a reprogramming factor and a cationic conjugate. Cationic conjugate herewith comprises a plurality of residues selected from amines, guanidines, amidines, N-containing heterocycles, or combinations thereof. In related embodiments, the cationic conjugate may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof. In preferred embodiments, the cationic conjuage is an oligomer selected from the group consisting of oligopeptide, oligoamide, cationically functionalized oligoether, cationically functionalized oligosaccharide, oligoamine, oligoethyleneimine, and the like, as well as combinations thereof. The oligomers may be oligopeptides where amino acid residues of the oligopeptide are capable of forming positive charges. In other embodiments, the oligopeptides may comprise 5 to 25 amino acids; preferably 5 to 15 amino acids; more preferably 5 to 10 amino acids.

(a) Conjugate with Cationic Lysine or Arginine Oligopeptides

In another aspect, the present invention provides fusion proteins comprising oligopeptides (polypeptides) where amino acid residues of the oligopeptide are capable of forming positive charges.cationic polypeptide such as, but not limited to Oct4-polylysine, Oct4-polyarginine, Sox2-polylysine, Sox2-polyarginine, c-Myc-polylysine, c-Myc-polyarginine, Klf4-polylysine, Klf4-polyarginine, and Nanog-polylysine and Nanog-polyarginine. One skill in the art would readily utilize other oligopeptides (polypeptides) comprising any natural or unnatural amino acid residues capable of forming positive charge, e.g. histidine, ornithine, homoarginine and the like. The conjugation and recombinant techniques are described in more detail below.

The present invention also provides fusion proteins that have a reprogramming factor linked to a hybrid cell penetrating peptide made up of both lysine and arginine amino acids and the like. Thus, the present invention also provides fusion proteins such as Oct4-polylysine/polyarginine, Sox2-polylysine/polyarginine, c-Myc-polylysine/polyarginine, Klf4-polylysine/polyarginine, and Nanog-polylysine/polyarginine.

Accordingly, a cationic oligopeptide or polypeptide of the present invention includes, but is not limited to, a contiguous string of lysine amino acids, or arginine amino acids, or a mixture of lysine and arginine amino acids, or the like. Thus, the present invention provides homopolymeric lysine peptides, homopolymeric arginine peptides, heteropolymeric lysine/arginine peptides, and the like, which may be fused, conjugated, ligated, or otherwise joined to a reprogramming factor protein. Furthermore, the present invention provides the fusion of the same or different cell penetrating peptide at one or both ends of the reprogramming factor protein. By “different,” is meant that two lysine-rich peptides may be different by virtue of the number of residues in each peptide, in addition to the amino acid composition of the peptides. Thus, the present invention provides the fusion of, for example, a 6-lysine peptide at the N-terminus of a reprogramming factor and a 9-lysine peptide at its C-terminus.

Accordingly, the length of a cationic lysine or arginine peptide of the present invention may be 5 amino acids in length, 6 amino acids in length, 7 amino acids in length, 8 amino acids in length, 9 amino acids in length, 10 amino acids in length, 11 amino acids in length, 12 amino acids in length, 13 amino acids in length, 14 amino acids in length, 15 amino acids in length, 16 amino acids in length, 17 amino acids in length, 18 amino acids in length, 19 amino acids in length, 20 amino acids in length, or more than 20 amino acids in length. The peptide may be between 5 and 10 amino acids in length. In one embodiment, the cell penetrating peptide comprises 9 residues, i.e., 9 contiguous lysine residues or 9 contiguous arginine residues.

In the case of hybrid hetero-cell penetrating peptides one or more lysines or arginines may reside within the longer arginine-rich or lysine-rich peptide, respectively. That is, a predominantly lysine-rich peptide may comprise one or more arginine residues; likewise, a predominantly arginine-rich peptide may comprise one or more lysine residues. In addition, a hetero-cell penetrating peptide of the present invention may comprises a string of contiguous lysine residues adjacent to a string of contiguous arginine residues. For instance, a hetero-cell penetrating peptide of the present invention may contain 9 lysine residues joined to 9 arginine residues. The present invention is not limited to any particular embodiment of cell penetrating peptide composition but may comprises various permutations and arrangements that help facilitate the delivery of a protein to which they are attached across a cell membrane and into the cell environment.

In this regard, a reprogramming factor/lysine-or-arginine cell penetrating peptide fusion protein of the present invention can be made in different ways, such as by chemical conjugation and recombinant expression methods.

(i) Chemical Conjugation

A reprogramming factor of the present invention, whether it be chemically synthesized, made recombinantly, or isolated from a cell extract or tissue, may be chemically conjugated to a cationic oligopeptide of the present invention. Standard techniques for conjugating one peptide to another are well known. See, for instance Kennerly S. Patrick, Chemistry of Peptide Synthesis, CRC; 1 edition (Aug. 12, 2005).

The present invention provides the conjugation of a cell penetrating peptide at the N-terminus of a reprogramming factor or at its C-terminus. Furthermore, the present invention provides the conjugation of the same or different cell penetrating peptide at both N- and C-termini of a reprogramming factor. See FIGS. 18 and 19 for exemplary embodiments of chemically-conjugated cell penetrating peptides and reprogramming factors.

As used herein, the term “amino acids” include the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to embrace both the (D) and (L) stereoisomers.

As used herein, the term “cationically functional monosaccharides” may include any amine-containing monosaccharide such as glucosamine, galactosamine and 2-amino-sialic acid. It may also include any natural or unnatural derivatized monosaccharides containing one or more functional groups that can form positive charge, e.g. amine and phosphorus containing groups.

As used herein, the term “cationically functionalized oligosaccharide” is an oligosaccharide comprising one or more “cationically functional monosaccharides.”

As used herein, the term “cationically functionalized ethylene glycols” may include any substituted ethylene glycols where the substituents comprise functional groups that can form positive charge, e.g. amine and phosphorus containing groups.

As used herein, the term “cationically functionalized oligoether” may include any substituted oligoether where the substituents comprise functional groups that can form positive charge, e.g. amine and phosphorus containing groups.

In another aspect, the linker may be selected from the group consisting of a disulfide linkage, a protected disulfide linkage, an ether linkage, a thioether linkage, a sulfoxide linkage, an amine linkage, a hydrazone linkage, a sulfonamide linkage, an urea linkage, a sulfonate linkage, an ester linkage, an amide linkage, a carbamate linkage, a dithiocarbamate linkage, and the like, as well as combinations thereof. The linkage can be prepared in a variety of ways, e.g. by functional group conversion at C or N terminus of a reprogramming factor protein (e.g. an amine linkage, a sulfonamide linkage, an ester linkage, an amide linkage) and/or by linkage of the side chain to appropriate linkers (e.g. a thioether linkage, a sulfoxide linkage, linkage, an ether linkage, an amine linkage, an ester linkage).

The linkers used in the present invention are selected based on the desired length of the linkers, the chemical property of the linkers and the chemistry employed for derivatization. Linkers with more than one possible orientation for attachment to reprogramming factor protein should be understood to embrace all possible orientations for attachment. For example, an ester linkage can be linked via hydroxy (—OC(O)—) or via oxo (—C(O)O—) moiety; a sulfonate linkage may be linked via hydroxy (—OS(O)₂—) or via mercapto (—S(O)₂O—) moiety; a thiocarbamate linkage may be linked via hydroxy (—OC(S)NH—) or via amino (—NHC(S)O—) moiety. Other suitable linkers for attachment of each cationic conjugate can be readily identified.

(ii) Cell Penetrating Peptides

Cell penetrating peptides (“CPPs”) are short peptides that facilitate cellular uptake of various molecular cargos. One function of CPPs is to deliver the cargo into cells, a process that commonly occurs through endocytosis, with the cargo delivered to the endosomes of living mammalian cells.

A “cargo” can be anything from small chemical molecules to nanosize particles and large fragments of DNA and proteins. Any such cargo can be co-joined to a CPP peptide either through chemical linkage via covalent bonds or through non-covalent interactions. Or a nucleotide sequence that encodes a CPP peptide can be engineered into or subcloned adjacent to a polynucleotide that encodes a cargo protein or nucleic acid sequence of interest. Thus expression of the entire sequence could produce, for example, a fusion protein comprising the cargo protein fused to the CPP peptide. Further below is a discussion of the use of CPPs for delivering oligonucleotide cargos to cells.

CPPs typically have an amino acid composition containing either a high relative abundance of positively charged amino acids such as lysine or arginine, or have sequences that contain an alternating pattern of polar/charged amino acids and non-polar, hydrophobic amino acids. In 1988, Frankel and Pabo found that the human immunodeficiency virus transactivator of transcription (HIV-TAT) protein can be delivered to cells using a CPP (Frankel et al., 1988a and Frankel et al., 1988b).

A CPP employed in accordance with one aspect of the invention may include 3 to 35 amino acids, preferably 5 to 25 amino acids, more preferably 10 to 25 amino acids, or even more preferably 15 to 25 amino acids. Thus, the present invention also contemplates encoding nucleotide sequences that encode these peptidic lengths of CPPs.

A CPP may also be chemically modified, such as prenylated near the C-terminus of the CPP. Prenylation is a post-translation modification resulting in the addition of a 15 (farneysyl) or 20 (geranylgeranyl) carbon isoprenoid chain on the peptide. A chemically modified CPP can be even shorter and still possess the cell penetrating property. Accordingly, a CPP, pursuant to another aspect of the invention, is a chemically modified CPP with 2 to 35 amino acids, preferably 5 to 25 amino acids, more preferably 10 to 25 amino acids, or even more preferably 15 to 25 amino acids.

A CPP suitable for carrying out one aspect of the invention may include at least one basic amino acid such as arginine, lysine and histidine. With respect to encoding DNA triplets (codons), arginine may be encoded by the sequences CGU, CGC, CGA, CGG, AGA, and AGG; lysine may be encoded by the sequences AAA and AAG; and histidine encoded by the sequences CAU and CAC. Accordingly, combinations of these codons can be designed to create particular or desirable CPPs as described next.

In another aspect, the CPP may include more, such as 2, 3, 4, 5, 6, 7, 8, 9, 10, or more such basic amino acids, or alternatively about 5%, 10%, 15%, 20%, 25%, 30%, 40%, 50% of the amino acids are basic amino acids. In one embodiment, the CPP contains at least two consecutive basic amino acids, or alternatively at least three, or at least five consecutive basic amino acids. In a particular aspect, the CPP includes at least two, three, four, or five consecutive arginine. In a further aspect, the CPP includes more arginine than lysine or histidine, or preferably includes more arginine than lysine and histidine combined.

CPPs may include acidic amino acids but the number of acidic amino acids should be smaller than the number of basic amino acids. In one embodiment, the CPP includes at most one acidic amino acid. In a preferred embodiment, the CPP does not include acidic amino acid. In a particular embodiment, a suitable CPP is the HIV-TAT peptide.

CPPs can be linked to a protein recombinantly, covalently or non-covalently. A recombinant protein having a CPP peptide can be prepared in bacteria, such as E. coli, a mammalian cell such as a human HEK293 cell, or any cell suitable for protein expression. Covalent and non-covalent methods have also been developed to form CPP/protein complexes. A CPP, Pep-1, has been shown to form a protein complex and proven effective for delivery (Kameyama et al., 2006 Bioconjugate Chem. 17:597-602).

CPPs also include cationic conjugates which also may be used to facilitate delivery of the proteins into the progenitor or stem cell. Cationic conjugates may include a plurality of residues including amines, guanidines, amidines, N-containing heterocycles, or combinations thereof. In related embodiments, the cationic conjugate may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof. The cationic conjugate also may be an oligomer including an oligopeptide, oligoamide, cationically functionalized oligoether, cationically functionalized oligosaccharide, oligoamine, oligoethyleneimine, and the like, as well as combinations thereof.

The oligomers may be oligopeptides where amino acid residues of the oligopeptide are capable of forming positive charges. The oligopeptides may contain 5 to 25 amino acids; preferably 5 to 15 amino acids; more preferably 5 to 10 cationic amino acids or other cationic subunits. Recombinant proteins anchoring CPP to the proteins can be generated to be used for delivery to neural progenitor cells or stem cells to prepare mature and functional DA neurons.

Accordingly, in one aspect, the invention provides a method for producing a neural cell from neural progenitor cells or stem cells by contacting a neural progenitor cell or neural stem cell with at least one protein of the Wnt1-Lmx1a signaling pathway selected from the group consisting of Wnt1, Lmx1b, Lmx1b, Otx2 and Pitx3 and at least one protein of the SHH-FoxA2 signaling pathway selected from the group consisting of SHH, FoxA2 and Nurr1 under conditions suitable for the proteins to penetrate the cells. Preferably, each of the proteins is attached to a CPP.

In some embodiments, the proteins comprise FoxA2, Lmx1a and/or Otx2, or alternatively include Nurr1, Pitx3 and/or Lmx1a, or alternatively include Nurr1, Pitx3, Lmx1a, FoxA2 and/or Otx2. In some embodiment, the neural cells can be further in contact with one or more of En1, En2 and/or Ngn2, which can be optionally attached to a CPP.

In another aspect, the invention provides modified polypeptides useful for producing neural cells from progenitor cells. The polypeptides include the various Wnt1-Lmx1a signaling pathway members (e.g., Wnt1, Lmx1a, Lmx1b, Otx2 and Pitx3) and the various SHH-FoxA2 signaling pathway members (e.g., SHH, FoxA2 and Nurr1), fused to CPP. In some embodiments, the CPP is fused to the C-terminus of the proteins either directly or through a linker (e.g., an amino acid or polymer linker). Suitable CPPs include, for example, the HIV TAT protein or any polycationic polypeptide or polymer (e.g., at least five consecutive arginine residues). One, two, three, four, five, or more of these polypeptides may be incorporated into a pharmaceutical formulation which itself may be administered to a patient, in a therapeutically effective amount, for the treatment or prevention of Parkinson's Disease. Thus, one embodiment of the present invention concerns providing to a cell a reprogramming factor selected from the group consisting of Oct4, Sox2, c-Myc, Klf4, Nanog, and Lin28, that is linked to a CPP peptide as described herein. Hence, the present invention contemplates conjugates such as Oct4-CPP, Sox2-CPP, c-Myc-CPP, Klf4-CPP, Nanog-CPP, and Lin28-CPP.

The trans-activating transcriptional activator (TAT) from human immunodeficiency virus 1 (HIV-1) is a CPP, which is able to deliver different proteins, such as horseradish peroxidase and RNase A across cell membrane into the cytoplasm in different cell lines. Wadia et al. (2004) Nat. Med. 10:310-15. Accordingly, in one aspect, a protein, such as Lmx1b, can be delivered to a neural precursor cell using TAT as a vehicle to increase the biological activity of Lmx1b in the cell.

Examples therefore of CPPs include the HIV-1 TAT sequence, e.g., Tat-(48-60) GRKKRRQRRRPPQ, and the homeodomain of the Drosophila homeoprotein Antennapaedia (penetratin residues 43-58) RQIKIWFQNRRMKWKK; and R8 (octo-arginine) RRRRRRRR. See Howl et al., Biochemical Society Transactions (2007), vol. 35, part 4, pp. 767-769, which is incorporated herein by reference. Howl reports that there are various chimeric CPP peptides that can be made which are capable of traversing the cell membrane, which may provide improved translocation properties compared to non-chimeric CPPs. For instance, chimeric CPPs may include peptides associated with tumor-homing peptides, nuclear localization sequences, protease-cleavable sites, integrin-binding RGD (Arg-Gly-Asp) sequences, sugars, lipids, and membrane-active peptides. Such chimeric CPPs may be useful for better targeting particular cell types, disease sites, and organelles.

As mentioned above, CPPs can also be used to deliver oligonucleotide cargoes to cells. Howl et al. (supra). Examples of nucleic acids that can benefit from attachment to a CPP include but are not limited to neutral peptide nucleic acids (PNAs), phosphorodiamidate morpholino oligomers (PMOs), and double-stranded RNA molecules, such as hairpin RNAs and duplexes used in RNA interference or as short interfering RNAs. Arginine-rich CPPs and R6-penetratin are useful for delivering PNAs and PMOs. A CPP peptide may be non-covalently linked to RNA molecules to improve cellular uptake of siRNA molecules.

(iii) Recombinant Fusion Proteins

A fusion protein of the present invention also may be made recombinantly. An expression cassette that expresses a reprogramming factor, as described in the preceding passages, may be further engineered to comprise a sequence that encodes one or more cationic lysine or arginine peptides. Codons that encode lysine include AAA and AAG. Codons that encode arginine include CGT, CGC, CGA, CGG, AGA, and AGG. Accordingly, permutations of these codons can be engineered into a polynucleotide sequence so that a particular cell penetrating peptide is expressed recombinantly. To produce a lysine peptide recombinantly, the present invention provides the integration of a polynucleotide that comprises a string of AAA codons, or AAG codons, or combinations of both AAA and AAG codons, into an expression cassette operably linked to the polynucleotide sequence encoding a particular reprogramming factor. The lysine polynucleotide sequence may be positioned at the 5′-end of the reprogramming factor sequence or at the 3′-end of the reprogramming factor sequence, or a lysine polynucleotide may be positioned at both ends of the factor sequence.

Similarly, an arginine-encoding polynucleotide may be made up of one or more CGT, CGC, CGA, CGG, AGA, and AGG codons or permutations thereof; and likewise positioned at the 5′-end or 3′-end of a reprogramming factor sequence in the expression cassette.

Hybrid hetero-lysine/arginine-encoding polynucleotide sequences can be made by combining different codons for each residue, and incorporating that sequence into the expression cassette.

Thus, an expression cassette of the present invention may comprise a polynucleotide sequence that, when expressed, produces a fusion protein comprising at least one cell penetrating peptide and a reprogramming factor.

The expression cassette may for instance comprise a promoter operably linked to an insert that comprises a polynucleotide sequence encoding a homo- or heter-cell penetrating peptide which is operably linked to a sequence encoding a reprogramming factor, which is operably linked to appropriate termination and polyadenylation sequences to facilitate timely and appropriate expression of that insert.

The recombinant expression cassette can be expressed in any of a number of available expression systems, such as in human cells, e.g., HEK cells, Chinese Hamster Ovary cells (CHO), insect expression systems, bacterial cell expression systems, or yeast expression systems.

(b) Combine with Other Cell Penetrating Peptides

The present invention is not limited to the creation of lysine- or arginine-specific fusion proteins with reprogramming factors. Other cell penetrating peptides may be chemically conjugated or recombinantly engineered onto a reprogramming factor of the present invention. Such other cell penetrating peptides includes, but is not limited to TAT peptides, Penetratin, VP22, and Buforin II.

Delivery of Reprogramming Factor Proteins to Cells

According to the present invention, one or more reprogramming factor proteins are added to cells which are to become iPS pluripotent cells. The reprogramming factor proteins can be isolated and purified from a cell expression system and those purified proteins added to the target cells; or the cellular extract of cells expressing the reprogramming factor proteins can be obtained (by lysing or otherwise breaking open the expression system cells). A reprogramming factor of the present invention may be covalently, non-covalently, or recombinantly linked to, joined to, fused to, or associated with any cell penetrating peptide (CPP), as described herein.

Accordingly, either the purified protein or protein extract can be added directly to the target cells. These techniques are well known. See for instance Example 3 below. Basically, to prepare cell protein extracts, cells are lysed in a lysis buffer, resuspended, and sonicated. The centrifuged sonicated material consists of a pellet of coarse cellular material and the supernatant the protein extract, which can then be removed and frozen until needed.

The target cells, e.g., differentiated somatic cells, can then be incubated with purified individual or multiple reprogramming factor proteins, or protein cellular extract(s) for a first incubation period of a number of hours per week for a number of weeks in a suitable medium, such as ES 1 medium described herein (see Example 4). ES1 medium is similar to regular media of MEF, but contains a little higher FBS and other ingredients and is useful for facilitating fibroblast-like cells to grow. The target cells can be incubated with the reprogramming factor protein(s) for about 5 hours per week, about 6 hours per week, about 7 hours per week, about 8 hours per week, about 9 hours per week, about 10 hours per week, about 11 hours per week, about 12 hours per week, or more than about 12 hours per week; and this cycle can be repeated for about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks, or more than about 12 weeks. In addition, the target cells can be incubated with the reprogramming factor protein(s) for about 0.1 hours per day, about 0.5 hours per day, about 1.0 hours per day, about 2.0 hours per day, about 3.0 hours per day about 4.0 hours per day, about 5.0 hours per day, about 6.0 hours per day, or more than about 6.0 hours per day; and this cycle can be repeated for about 5 days, about 6 days, about 7 days, about 8 days, about 9 days, about 10 days, about 11 days, about 12 days, about 13 days, about 14 days, about 15 days, or about 16 days, or more than about 16 days.

After that first incubation period, the cells can then be cultured on ES 2 medium (a mix of ES1 and ES3 media for culturing intermediate cells between fibroblast and iPS), for one, two, three, four, five, six, seven days, or more than seven days. In one embodiment the cells are incubated for a week in ES 2 medium. After then, the cells can be cultured with ES 3 Medium (for final establishment of iPS cells; same as normal ES media) for a period of time until iPS formation can be observed. See Example 4.

Identifying iPS Cells

Pluripotent stem cell development induced by introduction of one or more reprogramming factor proteins into a differentiated cell can be identified and tracked in a number of ways. For instance, the iPS cell can be analyzed for the presence of certain marker genes that are expressed in pluripotent cells. See Example 5 and the representative marker genes shown in Table 1. The pluripotent marker genes can be identified by RT-PCR of total RNA extracted from sample cells that have been exposed to the reprogramming factor protein(s). See Example 5. Alternatively, other standard molecular biology techniques, such as Northern blot and probe hybridization identification techniques can be used to determine whether a pluripotent-specific gene is expressed in the post-factor-treated cells of the present invention.

Treated cells of the present invention also can be assessed for pluripotent stem cell status based on bisulfite genomic sequencing. See Example 6. This technique measures methylation patterns associated with regulatory and promoter regions of a gene. The Nanog and Oct4 genes of pluripotent stem cells have a distinct methylation pattern and thus the identification of these methylation patterns in what used to be a differentiated cell is indicative of the change of the differentiated cell to a pluripotent cell.

Cells may also be fingerprinted to create a DNA fingerprint profile, such as by the amplification of tandem repeats and microsatellites, and use of standard fingerprinting techniques such as random amplified polymorphic DNAs and restriction fragment length polymorphisms, which produce a DNA pattern based upon cDNAs made from the RNAs expressed in the cell before and after exposure to the reprogramming factor protein(s) of the present invention. See Example 7.

Finally, protein profiles of cells treated with the reprogramming factor protein(s) of the present invention can be generated using Western Blotting techniques (Example 8) and antibody-based immunocytochemistry (Example 9) to identify the presence of pluripotent-specific proteins in cells exposed to the reprogramming factor(s).

The present invention is not limited to the use of only these particular methods of identifying iPS cells, and one or more of these methods can be used collectively to confirm the change of a differentiated or somatic cell to the status of a pluripotent stem cell.

Differentiation of iPS Cells

Pluripotent stem cells that have been created according to the present invention can then be cultured and grown into a specific type of cell. iPS cells can be dissociated and embryonic bodies (EBs) allowed to form for four days after plating of iPS cells in bacterial dishes in the ES medium without bFGF (which makes cells stop proliferating and start differentiating). EBs were allowed one day to attach to tissue culture dishes and neuronal precursor were then selected for by incubation in serum-free ITSFn medium for a month. ITSFn medium is DMEM/F12 (1:1) supplemented with insulin (5 ug/ml) transferring (50 ug/ml), selenium chloride (30 nM), and fibronectin (5 ug/ml). (Okabe et al., (1996) Mech Dev 59:89-102). Serum-free ITSFn medium is selectively good for neural precursor cells. Thereafter, various differentiated cell types are seen from EBs.

EBs are spherical multi-cellular aggregates that comprise a variety of cell populations. The processes of neuron, cardiac muscle and hematopoietic cell differentiation have been investigated using ES in vitro differentiation systems. See Schmitt et al., Genes Dev. 5: 728-740, 1991; Keller, Cur. Open. Cell Boil. 7: 862-869, 1995; Sanchez-Carpintero and Narbona, Rev. Neurol. 33: 47-53, 2001; and Bain et al., Dev. Boil. 196: 342-357: 1995, all of which are incorporated herein by reference.

By culturing in a medium supplemented with fibroblast growth factor (FGF)-2, embryonic stem cells transfected with hepatocyte nuclear factor (HNF)-3.beta. were differentiated from albumin-induced cells, and differentiated into hepatocytes. See Ishizaka et al., FEBS J. 16: 1444-1446, 2002, and Abe et al., Exp. Cell Res. 229: 27-34. 1996; and Miyashita et al., Cell Transplantation. 11: 429-434, 2002, all of which are incorporated herein by reference.

Mesenchymal stem cells (MSCs) multipotent, and they can differentiate into at least three lineages (osteogenic, chondrogenic, and adipogenic) when cultured under defined in vitro conditions. Previously attempts at differentiation of mature hepatocytes from adult BM including human MSCs have been reported. See Camper and Tilghman, Biotechnology 16, 81-87, 1991; Nahon, Biochimie. 69, 445-459, 1987; and Medvinsky and Smith, Nature 422, 823-825, 2003); and U.S. Pat. No. 7,332,336, all of which are incorporated herein by reference.

Genetic Disease and iPS Cells

The reprogramming factor fusion proteins of the present invention may be useful for administering to individuals to correct or treat certain genetic diseases. Genetic diseases such as adenosine deaminase deficiency-related severe combined immunodeficiency (ADA-SCID), Shwachman-Bodian-Diamond syndrome (SBDS), Gaucher disease (GD) type III, Duchenne (DMD) and Becker muscular dystrophy (BMD), Parkinson disease (PD), Huntington disease (HD), juvenile-onset, type 1 diabetes mellitus (JDM), Down syndrome (DS)/trisomy 21, and the carrier state of Lesch-Nyhan syndrome, may be treated by reprogramming those cells expressing the mutant phenotype into a undifferentiated state. Thus, the resultant disease-specific stem cells may prove helpful in obtaining normal and pathologic human tissue formation in vitro, thereby enabling disease investigation and drug development. See Park et al., Cell, 134, 877 (2008).

If iPS cells from patient have fundamental dysfunction, iPS cells or cells derived thereof can be further improved by genetic engineering or other methods so that they can be functional after transplantation. For example, see the paper by Jaenish (Science, 318: 1920-1923)

Similarly, the generation of iPS cells from an individual permits the large-scale production of the cell types affected by that individual's disease. These cells could in turn be used for disease modeling, drug discovery, and eventually autologous cell replacement therapies. See Dimos et al., Science, 321, 1218 (2008).

Accordingly, the present invention provides a “personalized medicine” approach to reprogramming an individual's genetically-diseased cells that can be re-differentiated into a desirable, and genetically-“correct,” genotype. Thus, the present invention permits the administration of fusion proteins of the present invention directly to an individual, where cells in vivo are reprogrammed; as well as the extraction of cells from an individual to which one or more fusion proteins of the present invention are delivered directly in vitro, whereupon the resultant undifferentiated cells are placed back into the individual, or are further treated and cultured in vitro to differentiate into a new cell type. That new cell type can then be reintroduced into the individual.

Alternatively, a permanent cell line could be made from the individual's cells and treated with one or more fusion proteins as described herein, and then that undifferentiated cell line used in screening assays to determine the effect of certain drugs, chemicals, compounds, and genetic expression studies to evaluate particular treatment schemes. Such regenerative treatments are therefore particularly amenable to the use of the iPS cells produced according to the present invention, such as regenerative treatments for neuropathy and cardiopathy.

Accordingly, somatic cells involved in diseases can be used to generate iPS cells by adding the reprogramming factor proteins disclosed herein to the somatic cells such that a healthy and replenishable source of new undifferentiated cells can be cultured into a “disease-free” cell line and reintroduced into the individual. A method for selecting induced pluripotent stem cells that appear in a medium according to the method of the present invention is not particularly limited, and a well-known means may be suitably employed, for example, a drug resistance gene or the like can be used as a marker gene to isolate induced pluripotent stem cells using drug resistance as an index.

Various media that can maintain undifferentiated state and pluripotency of ES cells and various media which cannot maintain such properties are known in this field, and induced pluripotent stem cells can be efficiently isolated by using a combination of appropriate media. Differentiation and proliferation abilities of isolated induced pluripotent stem cells can be easily confirmed by using confirmation means widely applied to ES cells. See US 2009/0047263, which is incorporated herein by reference.

Use of Protein-Induced iPS Cells for Drug Discovery

It also is possible to use any of the induced pluripotent stem cells of the present invention in screening assays for new therapeutic compounds and drugs and for subsequent toxicology testing of such substances and other compounds on the iPS cells of the present invention. Accordingly, the iPS cells of the present invention, which are created from the reprogramming effects of certain proteins on differentiated cells, are very useful as drug discovery tools. The iPS cells can be used in high-content screening (HCS) assays and used to study and identify cellular events and phenotypes resultant from exposure of the iPS cells to certain compounds and substances. Thus, particular parameters of drug performance such as toxicity and specificity can be established simultaneously in subpopulations of iPS cells created by the present invention. A “compound” or drug which is added to an iPS cell of the present invention can be a chemical, a drug, a therapeutic substance, a compound, a protein, a peptide, or a nucleic acid. With respect to the latter, an iPS cell can be engineered to express a DNA or RNA molecule and used as a host to identify genetic interactions that may prove to be useful targets for the design of particular therapeutics. For instance, an iPS cell can be useful as a host for sense RNA, antisense RNA, or RNA interference (RNAi) assays, to identify the phenotypic and molecular effect(s) of suppression of expression of one or more particular endogenous genes. The effectuating nucleic acid which brings about the silencing or downregulation of a gene in the iPS cell may therefore be a candidate for a gene based therapy. Thus, any compound, drug, or therapeutic substance can be administered to an iPS cell of the present invention and then the iPS cell monitored for any particular effects of the compound, drug, or substance on the phenotype or biochemical or molecular properties of the iPS cell. Additionally, a differentiated cell made from an iPS of the present invention also can be likewise treated with one or more various substances to determine the effect of that substance on the differentiated cell phenotype.

Accordingly, the iPS cells generated by the methods of the present invention can be useful for, but are not limited to, ADME/Tox assays, cytotoxicity assays, cell authentication screens, studies of apoptosis, cell signaling studies, determination of cell viability, imaging and immunological detection, kinase assays, cytokine assays, and protease assays.

One particular therapeutic use of the iPS cells generated by the methods of the present invention is their use in screening for anti-cancer drugs. One key mechanism in cancer concerns the abnormal activation or mutation of kinases. Kinase inhibitor drugs are an important class of targeted therapeutics with the clinical success of several kinase inhibitors including Gleevec, Sutent, and Sprycell. Thus an iPS cell of the present invention can be used to identify kinase inhibitors. Likewise, other classes of proteins and substances can be identified as therapeutic inhibitors of cancers and other diseases, as mentioned in the preceding subsection.

Cosmetics And Cosmeceuticals

The iPS cells generated by the methods of the present invention, as well as the extracts of those cells, cell-conditioned media, isolated fractions or particular proteins or mixtures of proteins from those iPS cells can be useful for biological, medical, and cosmetic purposes. With respect to the latter, stem cells are useful in improving cosmetic and therapeutic cosmetic applications. For instance, iPS cells of the present invention, cell-conditioned media and extracts can be used to treat wrinkles, rejuvenate and whiten skin, grow cartilage, and bone, thus making them useful tools in surgery, reconstructive procedures, and cosmetic surgery in procedures such as anti-aging or anti-wrinkle applications and various implantations, such as in breast implant surgery. Thus, the iPS cells of the present invention are particularly useful in regenerating skin from a patient. One beneficial application is therefore the use of the iPS cells created by the present invention for creating skin grafts that can be applied to burn victims, treatment of skin inflammation, and for healing or repairing wounds. See also U.S. Pat. No. 7,479,279, which is incorporated herein by reference.

A small amount of the individuals cells can be removed from the individual and treated with one or more reprogramming factors of the present invention to produce iPS cells which may then be used directly to treat certain conditions, such as those rectified by cosmetic procedures. Thus, the iPS cells of the present invention are a useful and more patient-friendly alternative to certain existing cosmetic procedures currently available, such as painful Botox injections.

Accordingly, iPS cells or extracts or isolates (e.g., proteins, RNAs, and other isolated cellular components), therefrom can be used for such treatments. In particular, cytokines can be isolated from the iPS cells of the present invention. Cytokines are useful signaling molecules that, generally-speaking, have autocrine, paracrine, and endocrine functions, which regulate immunity, inflammation, and hematopoiesis. The largest group of cytokines stimulates immune cell proliferation and differentiation. This group includes Interleukin 1 (IL-1), which activates T cells; IL-2, which stimulates proliferation of antigen-activated T and B cells; IL-4, IL-5, and IL-6, which stimulate proliferation and differentiation of B cells; Interferon gamma (IFNg), which activates macrophages; and IL-3, IL-7 and Granulocyte Monocyte Colony-Stimulating Factor (GM-CSF), which stimulate hematopoiesis. Accordingly, the naturally-synthesized cytokines of the iPS cells of the present invention can be isolated and formulated into creams, lotions, or other pharmaceutical and cosmetic medicaments and formulations and used to treat individuals.

In one embodiment, the invention provides cosmetic preparations (e g, skin creams, lotions, and solutions) which contain cell culture media in which the iPS cells of the present invention have been grown, or extracts or filtrates thereof (“conditioned cell media”).

These uses are exemplary uses of the iPS cells and their cell extracts and isolates of those cell extracts, for therapeutic and cosmetic treatments. Also exemplary are the following examples, which are provided purely to exemplify particular embodiments and methods and reagents of the present invention.

### EXAMPLES

### Example 1

### Cell Culture

Human newborn fibroblasts (HNF) were obtained from ATCC(CCL-117). HNF are cultured with Dulbecco's modified Minimal Essential Medium (DMEM, Invitrogen, Carlsbad, Calif.), supplemented with 2 mM L-glutamine (Invitrogen). 1 mM β-mercaptoethanol, 1× non-essential amino acids (NEAA; Invitrogen, Carlsbad, Calif.), 15% fetal bovine serum (FBS, Sigma-Aldrich, St. Louis, Mo.), 100 U/ml penicillin, 100 μg/ml streptomycin (Invitrogen)

When cultures were 10-20% confluent, the reprogramming experiment was initiated. Cultures were maintained at 37° C. in 5% CO₂, media changes were carried out every other day. For mouse embryonic fibroblast (MEF) isolation, uteri isolated from 13.5-day-pregnant CD1 mice were washed with phosphate-buffered saline (PBS). The head and visceral tissues were removed from isolated embryos. The remaining bodies were washed in fresh PBS, transferred into a 0.1 mM trypsin/1 mM EDTA solution, and incubated for 20 min. After incubation, MEF culture medium (DMEM containing 15% defined FBS) was added and pipetted up and down to dissociate cells. MEFs at passage two were used for the reprogramming experiment and passage one for feeder.

Induced pluripotent stem (iPS) cells were generated and maintained in human ES 3 medium (DMEM (Invitrogen, Carlsbad, Calif.), supplemented with 2 mM L-glutamine (Invitrogen). 1 mM β-mercaptoethanol, 1× non-essential amino acids (NEAA; Invitrogen, Carlsbad, Calif.), 20% knock-out serum replacement (KSR, Invitrogen, Carlsbad, Calif.), 100 U/ml penicillin, 100 μg/ml streptomycin (Invitrogen)).

Human ES cells and iPS cells were maintained on feeder layers of mitomycin C (10 μg/ml media, Sigma-Aldrich)-treated MEF cells. For picking and passaging, human iPS cells were washed once with ES medium and then mechanically handpicked (up to passage 35) or incubated with 0.1% collagenase type IV solution for 10 min. An appropriate volume of the medium was added, and the contents were transferred to a new dish onto MEF feeder cells. The split ratio was 1:1 (until passage 3) and after that, routinely 1:5. For feeder-free culture of iPS cells, the plate was coated with gelatin (StemCell Tech.).

### Example 2

### Plasmid Construction

Human cDNAs for OCT4, SOX2, KLF4, and c-MYC were amplified by RT-PCR from human ES poly(A⁺)RNA using the primers 5′-GGA TCC GAA TTC ATG GCG GGA CAC CTG GCT TCGG-3′ (SEQ ID NO.: 1) and 5′-AAA AAA GTC GAC gcg gcg tct gcg tct gcg gcg tct gcg GTT TGA ATG CAT GGG AGA GCC-3′ (SEQ ID NO.: 2) for human OCT4,5′-GGA TCC GAA TTC ATG TAC AAC ATG ATG GAG ACG G-3′ (SEQ ID NO.: 3) and 5′-AAA AAA CTC GAG gcg gcg tct gcg tct gcg gcg tct gcg CAT GTG CGA CAG GGG CAG TG-3′ (SEQ ID NO.: 4) for human SOX2,5′-GGA TCC GAA TTC ATG GCT GTC AGC GAC GCG CTG C-3′ (SEQ ID NO.: 5) and 5′-AAA AAA CTC GAG gcg gcg tct gcg tct gcg gcg tct gcg AAA GTG CCT CTT CAT GTG TAA GGC-3′ (SEQ ID NO.: 6) for human KLF4, and 5′-GGA TCC GAA TTC ATG CCC CTC AAC GTT AGC TTC AC-3′ (SEQ ID NO.: 7) and 5′-AAA AAA CTC GAG gcg gcg tct gcg tct gcg gcg tct gcg CGC ACA AGA GTT CCG TAG CTG TTC-3′ (SEQ ID NO.: 8) for human c-MYC (underlined nucleotides indicate region encoding the 9× arginines).

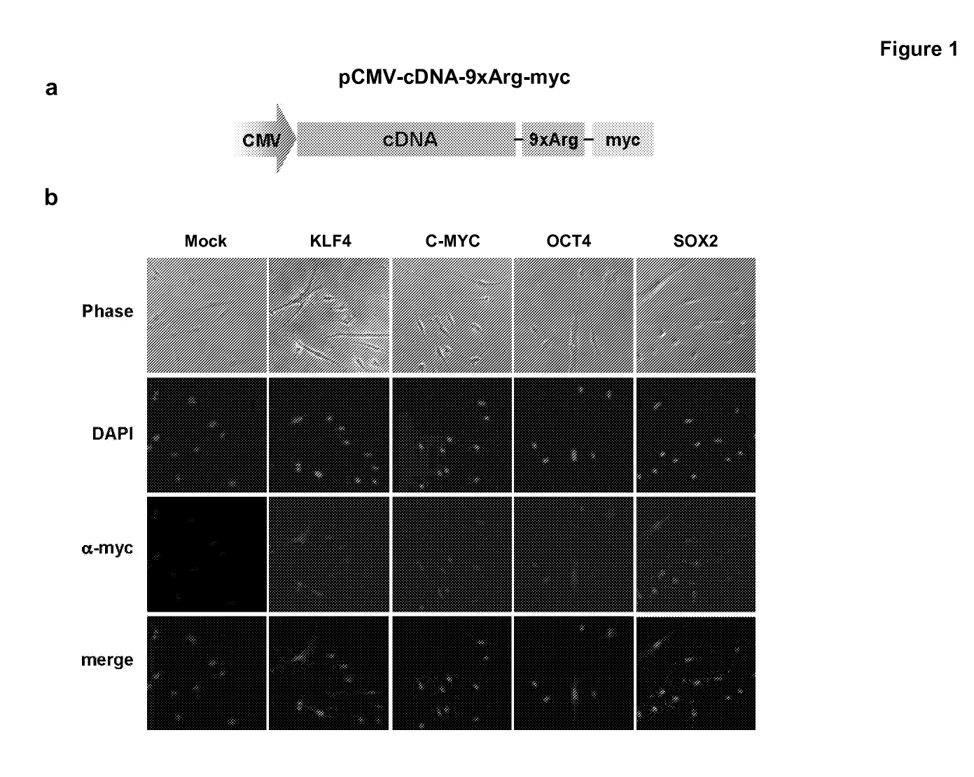

Amplified PCR products were digested with Eco RI and XhoI (or Sal I) and then cloned into the pcDNA3.1/myc-His A (Invitrogen), resulting in plasmid pCMV cDNA-9xArg-myc (FIG. 1). All plasmids used in transfection experiments were prepared by the EndoFree Plasmid Medi Kit (Qiagen). For construction of retrovirus vectors, 4 factors cDNAs were cloned into the retroviral expression vector pCL (Orbigen, San Diego, Calif., USA). Retrovirus was prepared using the 293GPG retroviral producer cell line as described in Ory et al. (Ory et al., (1996) PNAS 93:11400-11406). The correct cDNA insertion into the vector was confirmed by restriction digestion and sequence analyses.

### Example 3

### Making Protein Extracts

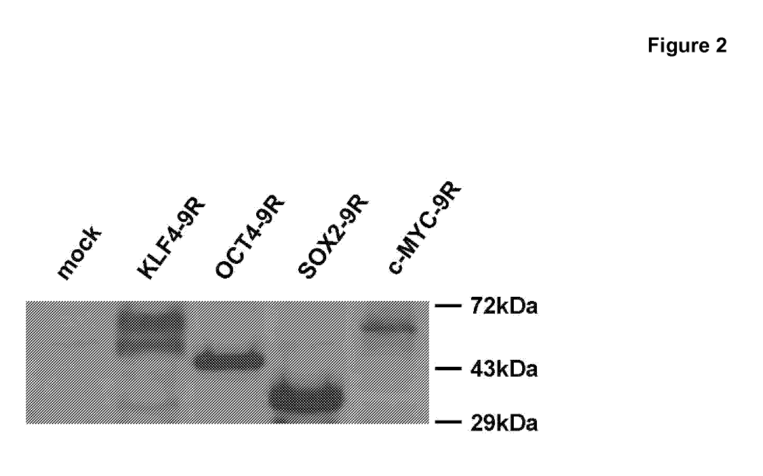

HEK 293 cells were maintained in Dulbecco's modified Eagle's medium (DMEM) supplemented with 10% fetal bovine serum containing 100 units/ml penicillin and 100 μg/ml streptomycin. A 2 μg plasmid DNA was transfected to approximately 4×10⁵ cells by Lipofectamine™ (Invitrogen). To establish cells stably expressing 4 factors, 1/1000 of cells 48 hours after transfection were seeded and further cultured in the presence of 500 μg/ml neomycin (G418 Sulfate, Clontech, Palo Alto, Calif.). An individual colony was isolated from neomycin-resistant colonies. The expression of 4 factors from neomycin-resistant colonies was determined by Western blot analysis (FIG. 2).

To prepare cell extracts, cells were washed in PBS and in cell lysis buffer (100 mM HEPES, pH 8.2, 50 mM NaCl, 5 mM MgCl₂, 1 mM dithiothreitol, and protease inhibitors), sedimented at 400 g, resuspended in 1 volume of cold cell lysis buffer, and incubated for 30-45 min on ice. Cells were sonicated on ice using a Labsonic-M pulse sonicator fitted with a 3-mm-diameter probe until all cells and nuclei were lysed, as judged by microscopy. The lysate was sedimented at 15,000 g for 15 min at 4° C. to pellet the coarse material. The supernatant was filtered with 0.2 μm membrane, aliquoted, frozen quickly in a dry ice, and stored at −80° C.

### Example 4

### Retroviral Infection, Protein Transduction and iPS Generation

For viral transduction, HEF cells cultured in vitro were incubated with the viral supernatant containing polybrene (hexadimethrine bromide; 1 μg/ml; Sigma) for 2-4 h. After infection, the cells were incubated five more days in normal culture media (DMEM with 15% defined-FBS). At six days of infection, cells were replated on gelatin-coated plate. Then virus-infected HEF cells are cultured DMEM with 15% defined-FBS for 24 hrs.

For protein transduction, HEF cells cultured in vitro were incubated with the 4 protein factors (each 12 μg/μl) for 8 hrs per week up to 6 weeks with ES 1 medium (DMEM supplemented with 2 mM L-glutamine, 1 mM β-mercaptoethanol, 1× non-essential amino acids, 20% FBS, 100 U/ml penicillin, 100 μg/ml streptomycin and 1500 U/ml LIF). After 6 weeks, the cells were dissociated and transferred onto MEF. Then, the cells are cultured with ES 2 medium (mixed medium (ratio; 1 (ES1 medium):3 (ES 3 medium)) for a week. After a week, the cells are cultured with ES 3 medium (DMEM supplemented with 2 mM L-glutamine, 1 mM β-mercaptoethanol, 1× non-essential amino acids, 20% KSR, 100 U/ml penicillin, 100 μg/ml streptomycin) until iPS formation.

### Example 5

### RT-PCR Analysis for Marker Genes

Total RNA was isolated from cells with Trizol reagent (Invitrogen). Five microgram of total RNA was used for reverse transcription reaction with SuperScript II (Invitrogen) and oligo-dT primer, according to the manufacturer's instructions. Real-time PCR analyses were performed in triplicate using SYBR green I using DNA engine Opticon™ (MJ Research, Waltham, Mass.) to analyze mRNA expression levels. Primer sets used to detect mRNA are shown in Table 1. Amplifications were performed in 25 μA containing 0.5 μM of each primer, 0.5×SYBR Green I (Molecular Probes, Oreg.), and 2 μl of cDNA. Fifty PCR cycles were performed with a temperature profile consisting of 95° C. for 30 sec, 55° C. for 30 sec, 72° C. for 30 sec, and 79° C. for 5 sec. The dissociation curve of each PCR product was determined to ensure that the observed fluorescent signals were only from specific PCR products. After each PCR cycle, the fluorescent signals were detected at 79° C. to melt primer dimers (Tms of all primer dimers used in this study were <76° C.). A standard curve was constructed using plasmid DNAs containing the GAPDH gene (from 10⁴ to 10⁹ molecules). The fluorescent signals from specific PCR products were normalized against that of the β-actin gene, and then relative values were calculated by setting the normalized value of control as 1. All reactions were repeated using more than three independent samples.

### Example 6

### Bisulfite Genomic Sequencing

Genomic DNA from cells was performed with the DNeasy Tissue Kit (Qiagen). Bisulfite treatment was done using the EpiTect Kit (Qiagen) following the manufacturer's instruction. The promoter regions of the human NANOG and OCT4 were amplified by PCR using primers published previously (Deb-Rinker et al., 2005 JBC 280:6257-6260; Freberg et al., 2007 MCB 18:1543-1553; Table 2). The resulting amplified PCR products were gel-purified, subcloned into the pGEM-T Easy vector (Promega) and sequenced.

### Example 7

### Fingerprinting Analysis

The regions of highly variable numbers of tandem repeats (VNTR) were amplified by PCR from genomic DNA in order to confirm the origin of iPS clones (Park et al., 2008 Nature 451:141). Amplified PCR products were subjected to 7% acrylamide gels. Primer sequences used in this study are listed in Table 3.

### Example 8

### Western Blotting

The cells were lysed with RIPA buffer containing of 50 mM Tris (pH7.5), 150 mM NaCl, 1% NP-40, 0.5% deoxycholic acid, and 0.1% SDS), supplemented with protease inhibitor cocktail (Roche) and mixed with an equal volume of a sodium dodecyl sulfate (SDS)-sample buffer consisting of 125 mM Tris (pH 6.8), 2% SDS, 15% glycerol, 5% β-mercaptoethanol, 0.05% bromophenol blue. Samples were subjected to SDS-polyacrylamide gel electrophoresis (PAGE) and transferred to a nitrocellulose membrane (Hybond-ECL, Amersham). After blocking, the membrane was incubated with 1:3000-dilution of an anti-myc antibody (Roche), followed by reaction with 1:300-dilution of a horseradish peroxidase-conjugated anti-mouse immunoglobulin G (IgG) antibody (Amersham). Detection was achieved using an enhanced-chemiluminescent substrate (Amersham).

### Example 9

### Alkaline Phosphatase Staining and Immunocytochemistry

Alkaline phosphatase (AP) staining was performed using the Alkaline phosphatase staining kit II (Vector Vector Laboratories, Burlingame, Calif.).

For immunocytochemistry, cells were fixed with 4% paraformaldehyde for 20 min at room temperature. After washing with PBS, the cells were treated with PBS containing 10% normal goat serum and 0.1% Triton X-100 for 45 min at room temperature. Primary antibodies included SSEA3 and 4 (monoclonal, 1:100, Developmental Studies Hybridoma Bank), Oct4 and Tra-1-60 (polyclonal, 1:300, SantaCruz Biotech., SantaCruz, Calif.), smooth muscle actin (SMA; monoclonal, 1:400, Dako, Glostrop, Denmark), anti-bIII tubulin (Tuj 1; monoclonal, 1:500, Covance, Richmond, Calif.), Desmin (polyclonal, 1:500, DAKO), Sox17 (monoclonal, 1:200, SantaCruz Biotech.), tyrosin hydroxylase (TH; polyclonal, 1:1,000, Pel-Freez, Rogers, Ark.), nestin (monoclonal, 1:1,000, BD Sciences, Franklin Lakes, N.J.). For detection of primary antibodies, fluorescence-labeled (Alexa fluor 488 or 568; Molecular Probes, Eugene, Oreg.) secondary antibodies were used according to the specifications of the manufacturer. Cells were mounted using Vectashield containing 4′,6-diamidino-2-phenylindole (DAPI; Vector Lab.) and analyzed by fluorescent microscopy.

### Example 10

### In Vitro Differentiation of iPS Cells

iPS cells were dissociated and EBs were allowed to form for four days after plating of iPS cells in bacterial dishes in the ES medium without nFGF. EBs were allowed one day to attach to tissue culture dishes and neuronal precursor were then selected for by incubation in serum-free ITSFn medium for a month. Thereafter, various differentiated cell types are seen from EBs.

### Example 11

### Teratoma Formation

Protein-derived Human iPS cells (clone 1 and 2) were suspended in DMEM containing 10% FBS. Nude mice were anesthetized with diethyl ether. We injected the cell suspension under the kidney capsule. Four weeks after the injection, tumors were surgically dissected from the mice. Samples were weighed, fixed in PBS containing 4% formaldehyde, and embedded in paraffin. Sections were stained with hematoxylin and eosin.

### Example 12

### Direct Delivery of Recombinant Reprogramming Proteins to Human Somatic Cells

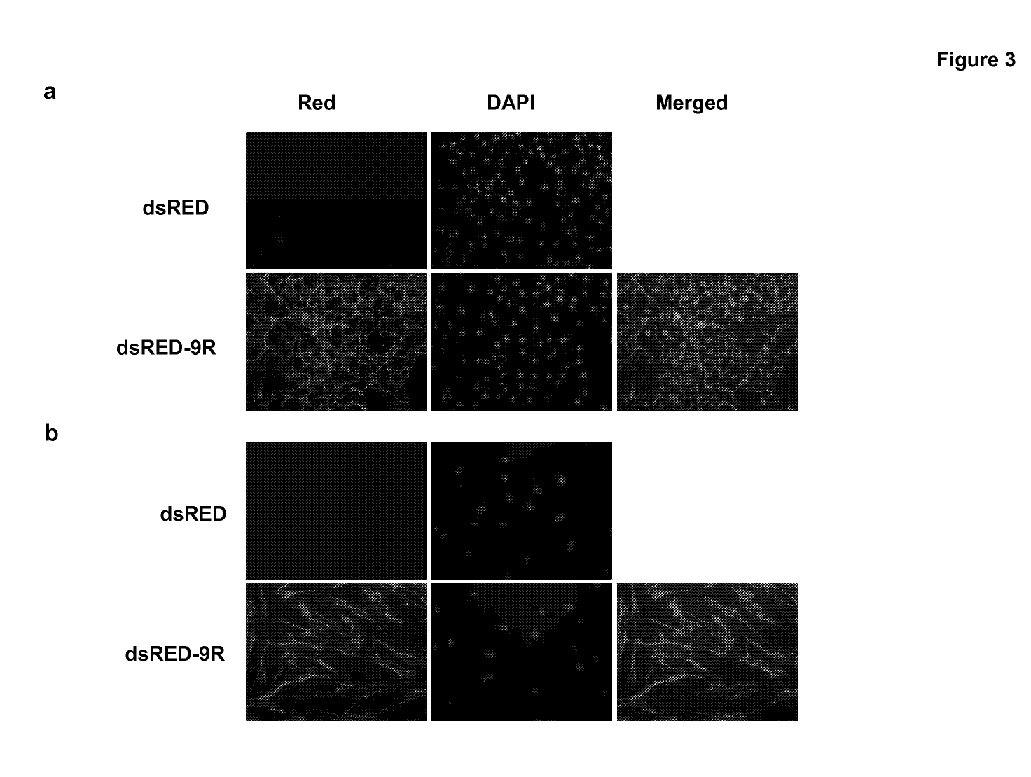

To address whether 9R-anchored reprogramming proteins can directly reprogram human somatic cells, human newborn fibroblasts (HNF) were chosen from the American Type Culture Collection (catalog number SCRC-1041) as targets. First, cell penetration was tested using the red fluorescent protein (RFP) fused with a nine-arginine peptide (RFP-9R). Indeed, RFP-9R was very efficiently delivered into both COS7 and HNF cells within several hours, even when treated along with whole cell extracts (FIG. 3). Next stable HEK293 cell lines were generated that can express each of the four human reprogramming factors (Oct4, Sox2, Klf4, and c-myc) fused with 9R and the myc tag (FIG. 1). Prominent expression of these proteins in stable HEK 293 cell lines was confirmed by Western blotting and immunocytochemical analyses (FIG. 1 and FIG. 3). When HNFs were treated with cell extracts from stable HEK 293 cell lines, efficient intracellular translocation of each recombinant protein was observed within 8 hours (FIG. 1). Notably, in contrast to RFP-9R, which was translocated to the cytoplasm, it appeared that most recombinant reprogramming proteins were translocated to the nucleus while some remained in the cytoplasm (FIG. 1 and FIG. 2).

### Example 13

### Generation of Hips Cell Lines, P-hiPS01 and P-hiPS02, by Direct Protein Delivery

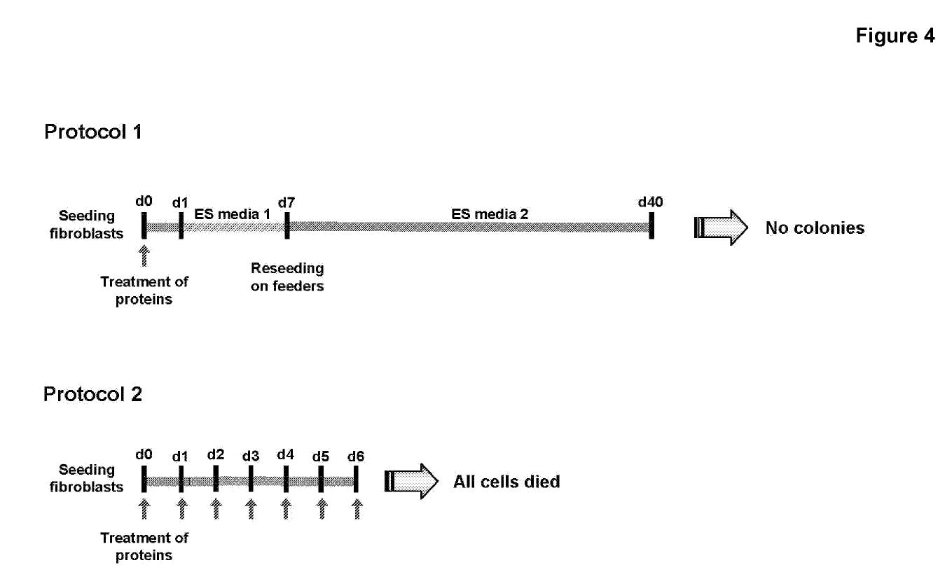

HNF cells (5×10⁵) were treated with combined total extracts of four stable HEK 293 cell lines for 16 hours. See Protocol 1 in FIG. 4. After washing, cells were incubated for 6 days in ES Media 1 (DMEM supplemented with 2 mM L-glutamine, 1 mM β-mercaptoethanol, 1× non-essential amino acids, 20% FBS, 100 U/ml penicillin, 100 μg/ml streptomycin and 1500 U/ml LIF). Those cells were then transferred onto mouse embryonic feeders (MEF). These transferred cells were incubated with ES Media 2 (mixed medium (ratio; 1 (ES1 medium):3 (ES 3 medium)) for up to 4 weeks but no reprogrammed colonies were identified.

The same total extracts were then treated for 16 hours followed by washing and incubation with ES Media 1 for 8 hours every day for 6 days (Protocol 2 in FIG. 4). At day 7, most cells did not survive, and no colony was formed after further incubation on MEF.

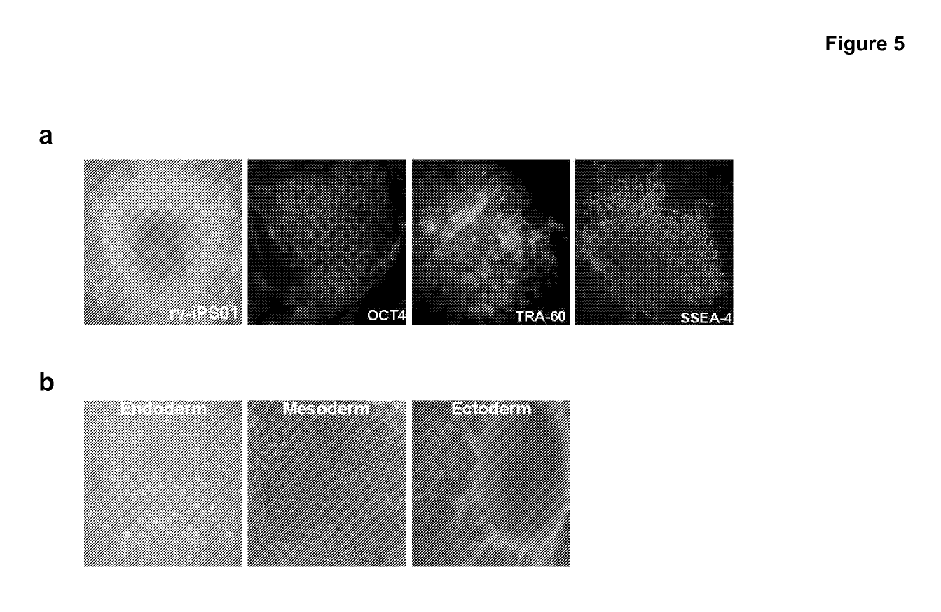

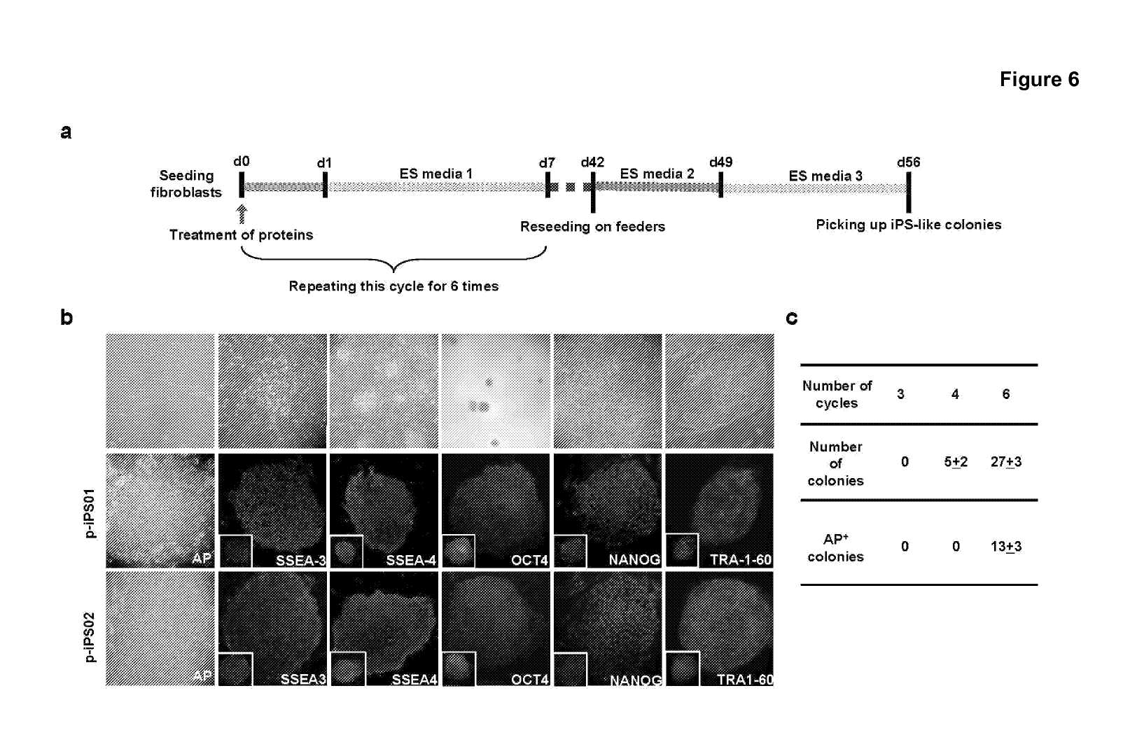

When viral or other DNA-based methods are used to express reprogramming factors, the host gene expression machinery should be able to continuously provide reprogramming factors. Thus, a repeated protein treatment cycle was tested to determine if this exposure regime would yield hiPS cells; accordingly this protocol required 16 hr protein treatment followed by 6 day incubation in ES Media 1. After treatment, cells were washed with fresh media and further incubated at 37 C for 6 days with daily media change. See FIG. 6. Indeed, after three or four rounds, several colonies with iPS-like morphology started to form (FIGS. 5 and 6).

When this “repetitive” protocol was further repeated, the number of iPS-like colonies significantly increased and approximately half of these colonies were AP-positive starting from the 6^{th} cycle (FIGS. 5 and 6). These AP-positive colonies with iPS-like morphology were picked and transferred onto the MEF in the presence of ES Media 2 and Media 3 (DMEM (Invitrogen, Carlsbad, Calif.), supplemented with 2 mM L-glutamine (Invitrogen). 1 mM β-mercaptoethanol, 1× non-essential amino acids (NEAA; Invitrogen, Carlsbad, Calif.), 20% knock-out serum replacement (KSR, Invitrogen, Carlsbad, Calif.), 100 U/ml penicillin, 100 μg/ml streptomycin (Invitrogen)), for 7 days each.

At this point, approximately 30 colonies with iPS-like morphology were formed and about half of them were AP-positive. Five iPS-like colonies were subsequently picked and two of them (p-hiPS01 and p-hiPS02) have been maintained and characterized. Both p-hiPS01 and p-hiPS02 have been passaged more than 35 times to date (FIG. 6). These two cell lines exhibited morphology similar to that of human ES cells, characterized by large nuclei and scant cytoplasm (FIG. 6). Overall, the establishment of these iPS-like colonies took approximately 8 weeks at an efficiency of iPS generation of about 0.001% of input cells; FIG. 5, compared to virus-based gene expression protocols (about 0.1 to 0.01%).

### Example 14

### Characterization of protein-induced hiPS cell Lines

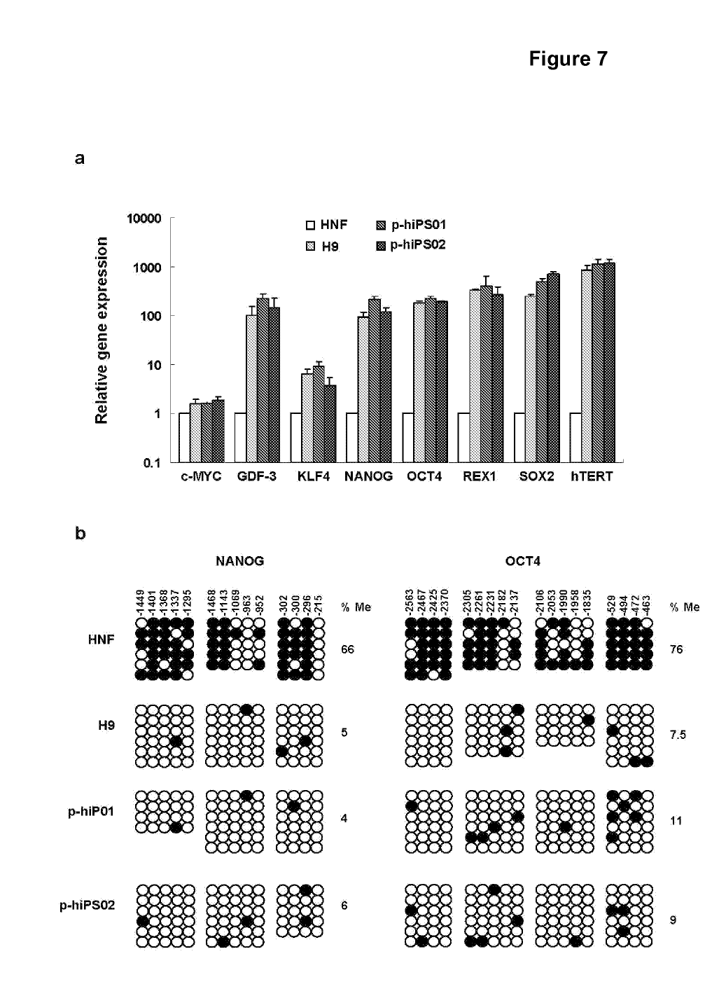

address whether these p-hiPS clones have hES cells' cellular properties we examined them for expression of ES markers. As shown in FIG. 6, both clones prominently expressed ES marker proteins such as AP, Oct4, Nanog, tumor-related antigen (Tra)1-60, stage-specific embryonic antigen (SSEA) 3 and SSEA 4. Furthermore, quantitative reverse transcription PCR (qRT-PCR) analysis demonstrated that these p-hiPS clones express mRNAs of ES cell markers, including OCT4, NANOG, SOX2, reduced expression 1 (REX1), growth and differentiation factor 3 (GDF3), telomerase reverse transcriptase (hTERT) at levels that were dramatically higher than those of HNF cells (up-to 100-fold) (FIG. 7). Induced levels of these ES markers in p-hiPS cells were comparable to those of the H9 hES line (FIG. 7). In contrast, expression levels of KLF4 and c-MYC in p-hiPS cells were only modestly higher than those of HNF cells. These expression patterns of various ES markers were indistinguishable from those of H9 cells and thus strongly suggest that faithful epigenetic reprogramming occurred in these p-hiPS cells. In support of this, bisulfite sequencing analyses showed that the promoter regions of the pluripotency genes, NANOG and OCT4, were greatly demethylated in both p-hiPS lines and hES H9 line, whereas these regions were densely methylated in the parental HNF cells (FIG. 7).

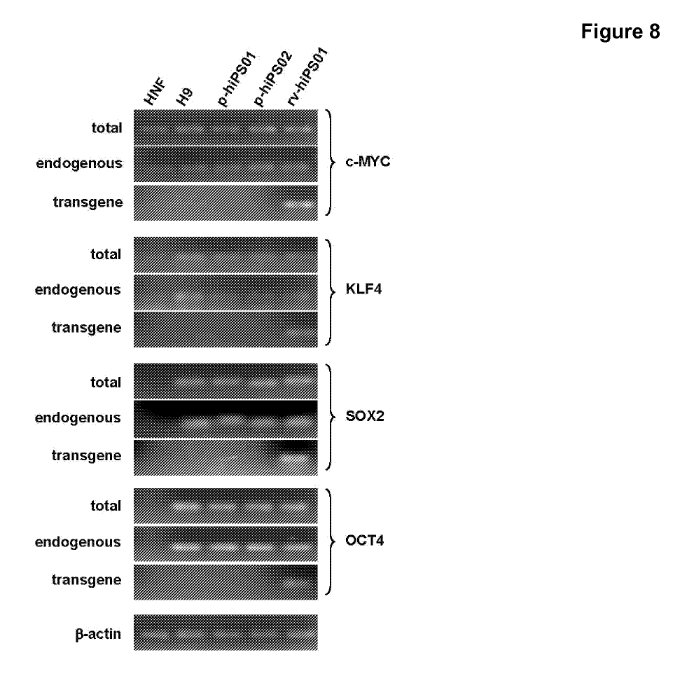

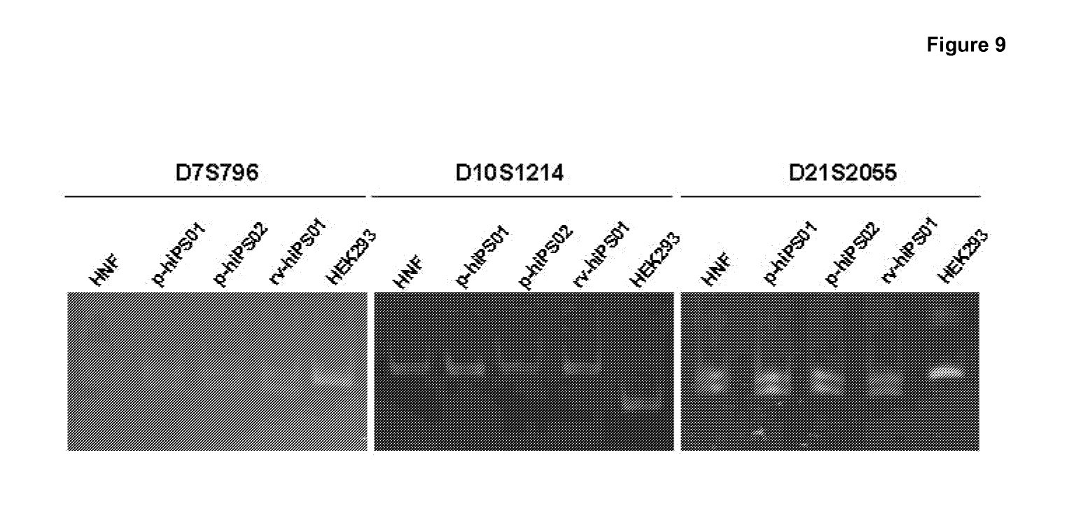

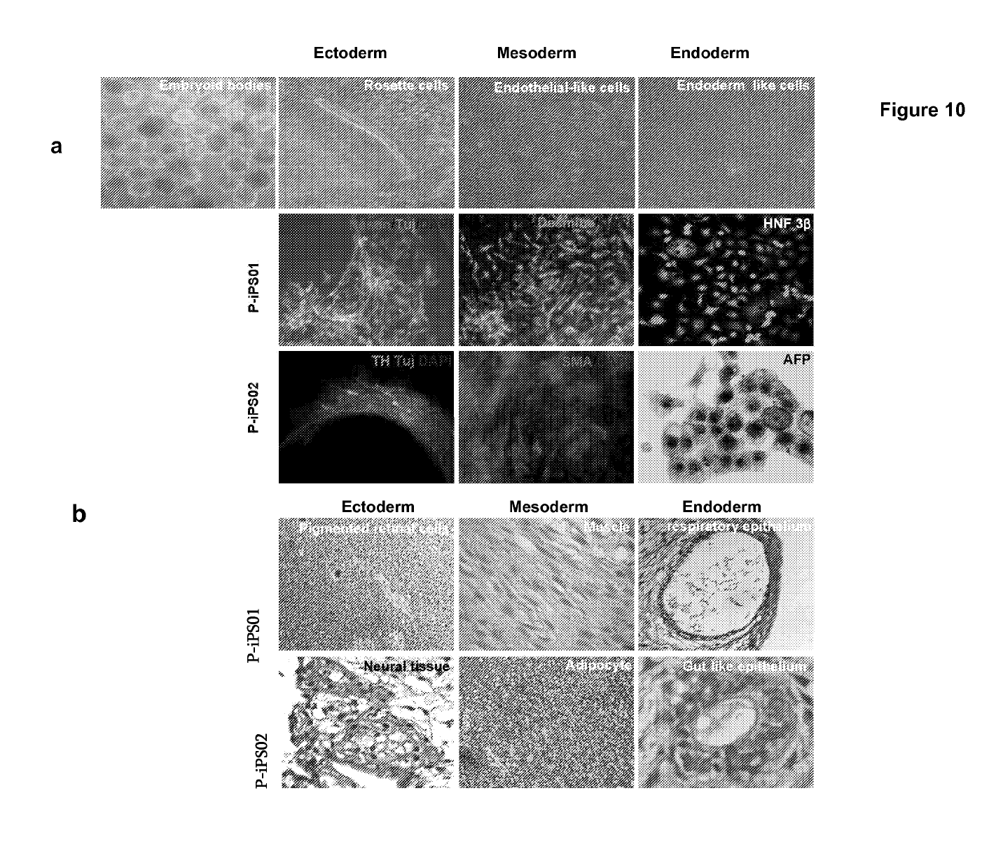

Human iPS lines were also generated from the same HNFs using the retroviral vectors expressing the same four reprogramming factors (Takahashi et al., 2007 Cell 131:861; Yu et al., 2007 Science 318:1917; Park et al., 2008 Nature 451:141), which satisfied the same criteria of iPS cells as described in this study (FIG. 8) One of them (rv-hiPS01) was used as a control (FIG. 9). Since hES H9 and rv-hiPS01 lines were used as control lines, it was important to rule out the possibility that the p-hiPS cells were derived from these contaminating cells. RT-PCR analyses detected chromosomal integrations of the transgenes in rv-hiPS01 cells, but not in p-hiPS01 and p-hiPS02 cell lines (FIG. 9). Furthermore, DNA fingerprinting analysis demonstrated that the patterns of both p-hiPS lines and rv-hiPS01 cells are identical to parental HNF cells, but are different from those of hES H9 cells, thus confirming that both p-hiPS lines are derived from HNF cells (ATCC cat no. SCRC1071). Furthermore, both p-hiPS lines exhibited the normal karyotype of 46XX at least for 35 passages (FIG. 10).

Embryoid body (EB) formation was then next induced from these p-hiPS cells by suspension culture (FIG. 10). After 8 days, well-formed EB structures were generated from both p-iPS clones. When these EB-like structures were incubated on gelatin-coated tissue culture plates in ITSFn media for 15 to 25 days, they differentiated to various cell morphologies of all three germ layers, including neural cells, muscle cells, and endoderm-like cells (FIG. 10). Immunocytochemical analyses demonstrated the existence of different cell types positive for SRY-box containing gene 17 (SOX17, endoderm marker), smooth muscle actin (SMA, mesoderm marker), desmin (mesoderm marker), Tuj 1 (ectoderm marker), nestin (ectoderm marker), and tyrosine hydroxylase (TH, ectoderm marker). In addition, transplantation of these p-hiPS clones into nude mice kidney capsules generated teratomas after 6 to 8 weeks. These teratomas contained tissues of all three germ layers including neural tissues (ectoderm), epidermal tissues (ectoderm), striated muscle (mesoderm), adipose tissue (mesoderm), respiratory epithelium (endoderm) and cornea-like epithelial tissues (endoderm) (FIG. 10), showing that both p-hiPS clones exhibit pluripotency both in vitro and in vivo.

### Tables

TABLE 1
Primer sequences for quantitative RT- PCR

| | Sense primer | SEQ ID | Antiense primer | SEQ ID |
|---|---|---|---|---|
| Gene | (5′ to 3′) | NO: | (5′ to 3′) | NO: |
| c-Myc | ACTCTGAGGAGGAACAAGAA | 9 | TGGAGACGTGGCACCTCTT | 10 |
| Klf4 | TCTCAAGGCACACCTGCGAA | 11 | TAGTGCCTGGTCAGTTCATC | 12 |
| Oct4 | GACAGGGGGAGGGGAGGAGCTAGG | 13 | CTTCCCTCCAACCAGTTGCCCCAAAC | 14 |
| Sox2 | AGCTACAGCATGATGCAGGA | 15 | GGTCATGGAGTTGTACTGCA | 16 |
| Nanog | TGAACCTCAGCTACAAACAG | 17 | TGGTGGTAGGAAGAGTAAAG | 18 |
| hTERT | TGTGCACCAACATCTACAAG | 19 | GCGTTCTTGGCTTTCAGGAT | 20 |
| Rex1 | TCGCTGAGCTGAAACAAATG | 21 | CCCTTCTTGAAGGTTTACAC | 22 |
| Gdf3 | AAATGTTTGTGTTGCGGTCA | 23 | TCTGGCACAGGTGTCTTCAG | 24 |
| β-actin | CTGGCACCACACCTTCTACAATG | 25 | AATGTCACGCACGATTTCCCGC | 26 |
| GAPDH | CAAAGTTGTCATGGATGACC | 27 | CCATGGAGAAGGCTGGGG | 28 |

TABLE 2
Primer sequences for bisulfite sequencing

| | | SEQ | | SEQ |
|---|---|---|---|---|
| | | ID | | ID |
| Gene | Sense primer (5′ to 3′) | NO: | Antiense primer (5′ to 3′) | NO: |
| Oct4-2 | TTAGGAAAATGGGTAGTAGGGATTT | 29 | TACCCAAAAAACAAATAAATTATAAAACCT | 30 |
| Oct4-3 | ATTTGTTTTTTGGGTAGTTAAAGGT | 31 | CCAACTATCTTCATCTTAATAACATCC | 32 |
| Oct4-4 | GGATGTTATTAAGATGAAGATAGTTGG | 33 | CCTAAACTCCCCTTCAAAATCTATT | 34 |
| OCT4-7 | TAGTTGGGATGTGTAGAGTTTGAGA | 35 | TAAACCAAAACAATCCTTCTACTCC | 36 |
| Nanog-1 | AGAGATAGGAGGGTAAGTTTTTTTT | 37 | ACTCCCACACAAACTAACTTTTATTC | 38 |
| Nanog-2 | GAGTTAAAGAGTTTTGTTTTTAAAAATTAT | 39 | TCCCAAATCTAATAATTTATCATATCTTTC | 40 |
| Nanog-3 | TTAATTTATTGGGATTATAGGGGTG | 41 | AACAACAAAACCTAAAAACAAACC | 42 |

TABLE 3
Primer sequencing for fingerprinting

| | Sense primer | SEQ ID | Antiense primer | SEQ ID |
|---|---|---|---|---|
| Gene | (5′ to 3′) | NO: | (5′ to 3′) | NO: |
| D7S796 | TTTTGGTATTGGCCATCCTA | 43 | GAAAGGAACAGAGAGACAGGG | 44 |
| D1051214 | ATTGCCCCAAAACTTTTTTG | 45 | TTGAAGACCAGTCTGGGAAG | 46 |
| D2152055 | AACAGAACCAATAGGCTATCTATC | 47 | TACAGTAAATCACTTGGTAGGAGA | 48 |

1. A method for producing pluripotent stem cells, comprising contacting differentiated cells with at least one reprogramming factor protein wherein the reprogramming factor protein(s) causes the differentiated cell to dedifferentiate.

2. The method of claim 1, wherein the reprogramming factor is selected from the group consisting of Oct4, Sox2, c-Myc, Klf4, Nanog, and Lin28.

3. The method of claim 1, wherein the reprogramming factor is linked to a cell penetrating peptide, wherein the reprogramming factor is chemically conjugated to the cell penetrating peptide or is recombinantly linked to the cell penetrating peptide as a fusion protein.

4. The method of claim 2, wherein the cell penetrating peptide is a peptide that comprises (a) at least nine contiguous lysine amino acid resides, (b) at least nine contiguous arginine amino acid residues; (c) at least nine residues of a mixture of lysine and arginine amino acids, or (d) the HIV-TAT protein or a fragment thereof.

5. The method of claim 1, wherein the at least one reprogramming factor is introduced into the differentiated cells in vitro.

6. The method of claim 1, wherein the at least one reprogramming factor is introduced into the differentiated cells in vivo.

7. The method of claim 1, comprising contacting said differentiated cells with an Oct4 reprogramming factor protein, wherein the Oct4 reprogramming factor protein comprises at least nine contiguous lysine resides or at least nine contiguous arginine residues, and wherein the presence of the Oct4 reprogramming factor protein in the differentiated cells causes the differentiated cells to become a pluripotent stem cells.

8. The method of claim 1, comprising contacting said differentiated cells with a Sox2 reprogramming factor protein, wherein the Sox2 reprogramming factor protein comprises at least nine contiguous lysine resides or at least nine contiguous arginine residues, and wherein the presence of the Sox2 reprogramming factor protein in the differentiated cells causes the differentiated cell to become a pluripotent stem cells.

9. The method of claim 1, comprising contacting said differentiated cells with a c-Myc reprogramming factor protein, wherein the c-Myc protein comprises at least nine contiguous lysine resides or at least nine contiguous arginine residues, and wherein the presence of the c-Myc reprogramming factor protein in the differentiated cells causes the differentiated cells to become a pluripotent stem cells.

10. The method of claim 1, comprising contacting said differentiated cells with a Klf4 reprogramming factor protein, wherein the Klf4 reprogramming factor protein comprises at least nine contiguous lysine resides or at least nine contiguous arginine residues, and wherein the presence of the Klf4 reprogramming factor protein in the differentiated cells causes the differentiated cell to become a pluripotent stem cells.

11. The method of claim 1, comprising contacting said differentiated cells with an Oct4 conjugate, a Sox2 conjugate, a c-Myc conjugate, and a Klf4 conjugate, wherein each of said conjugates comprises a cell penetrating peptide, wherein said conjugates cause said differentiated cells to become a pluripotent stem cells.

12. The method of claim 11, wherein the cell penetrating peptide comprises a plurality of substituents selected from amines, guanidines, amidines, N-containing heterocycles, or combinations thereof.

13. The method of claim 11, wherein the cell penetrating peptide comprises a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof.

14. The method of claim 13, wherein the cell penetrating peptide is an oligomer selected from the group consisting of oligopeptide, oligoamide, cationically functionalized oligoether, cationically functionalized oligosaccharide, oligoamine, oligoethyleneimine, and combinations thereof.

15. The method of claim 14, wherein the oligomer is an oligopeptide.

16. The method of claim 15, wherein substantially all of the amino acid residues of the oligopeptide are capable of forming positive charges.

17. The method of claim 16, wherein said oligopeptide comprises 5 to 15 amino acids.

18. The method of claim 17, wherein said oligopeptide comprises 5 to 10 amino acids.

19. The method of claim 15, wherein said oligopeptide comprises at least nine contiguous lysine resides, at least nine contiguous arginine residues; or combinations thereof.

20. The method of claim 11, wherein said cells are differentiated somatic cells.

21. The method of claim 11, wherein the reprogramming factor conjugate protein is delivered to the cells in vitro or in vivo.

22. The method of claim 2, wherein said differentiated cells are contacted with at least four reprogramming factor proteins.

23. The method of claim 22, wherein said at least four reprogramming factor proteins comprise Oct4, Sox2, c-Myc, and Klf4.

24. The method of claim 23, wherein each of said Oct4, Sox2, c-Myc, and Klf4 is linked to a cell penetrating peptide as a fusion protein.

25. The method of claim 24, wherein each of the cell penetrating peptides is independently a peptide that comprises (a) at least nine contiguous lysine amino acid resides, (b) at least nine contiguous arginine amino acid residues; (c) at least nine residues of a mixture of lysine and arginine amino acids; or (d) the HIV-TAT protein or a fragment thereof.

26-46. (canceled)

47. An induced pluripotent stem cell produced from the method of claim 1.

48. A differentiated cell produced from an induced pluripotent stem cell of claim 47.

49. The differentiated cell of claim 48 wherein the differentiated cell is produced by (A) producing embryonic bodies from the induced pluripotent stem cell made from the method of claim 1, and (2) incubating the embryonic bodies on ITSFn media, wherein the embryonic bodies differentiate into the cellular morphology of at least one germ layer selected from the group consisting of endoderm germ later cells, mesoderm germ layer cells, ectoderm germ layer cells.

50-57. (canceled)

58. A pharmaceutical composition, comprising a cell of claim 47.

59. A pharmaceutical composition, comprising a cell of claim 48.

60. A cosmetic preparation comprising conditioned cell media which has previously supported growth of the cells of claim 47.

61. The cosmetic preparation of claim 60, wherein said preparation is a cream.

62. The method of claim 1, further comprising contacting the differentiated cells with at least one of an inhibitor of p53, p16(Ink4a), and p19(Arf), ERas, ECAT15-2, Tell, and beta-catenin, ECAT1, Esg1, Dnmt3L, ECAT8, Gdf3, Sox15, ECAT15-1, Fthl17, Sal14, Rex1, UTF1, Stella, Stat3, and Grb2.
